(19) United States
(12) Patent Application Publication
(10) Pub. No.: US 2015/0025031 A1
(43) Pub. Date: Jan. 22, 2015

(54) METABOLIC DOWNREGULATION FOR CELL SURVIVAL

(71) Applicant: WAKE FOREST UNIVERSITY HEALTH SCIENCES, WINSTON SALEM, NC (US)

(72) Inventors: James Yoo, Winston Salem, NC (US); Sang Jin Lee, Winston Salem, NC (US); Jachyun Kim, Winston Salem, NC (US); Anthony Atala, Winston Salem, NC (US)

(21) Appl. No.: 14/382,921

(22) PCT Filed: Mar. 5, 2013
(86) PCT No.: PCT/US13/29025

§ 371 Date: Sep. 4, 2014

Related U.S. Application Data
(60) Provisional application No. 61/606,698, filed on Mar. 5, 2012.

(51) Int. Cl. A61K 31/7076

(52) U.S. Cl. CPC A61K 31/7076
USPC 514/45; 435/375

(57) ABSTRACT

The present invention provides a system and method of maintaining and/or increasing cell viability by downregulating cellular metabolic rate under hypoxic conditions, wherein the availability of adenosine or derivatives thereof in the cell is increased and/or prolonged. The present invention also relates to a system and method of prolonging the survival of implanted cells that are under hypoxic condition until host neovascularization is achieved, wherein the availability of adenosine or derivatives thereof in the cell is increased and/or prolonged. The present invention also provides a system and method of maintaining and/or increasing cell viability by downregulating cellular metabolic rate under hypoxic conditions, wherein at least one purine metabolism enzyme inhibitor is applied to the cell.

### CROSS-REFERENCE TO RELATED APPLICATION

This application claims priority to U.S. Provisional Application Ser. No. 61/606,698, filed Mar. 5, 2012, the contents of which are incorporated by reference herein in their entirety.

### BACKGROUND OF THE INVENTION

Building a clinically relevant sized tissue or organ using cells requires maintenance of viable cells until host vasculature is established and integrated into the implanted engineered constructs. Tissue engineering (TE) generally includes use of a scaffold that provides an architecture on which seeded cells are matured into tissues and organs. One of the foremost challenges in TE is the limitation imposed on oxygen supply immediately following implantation of the cell-scaffold construct. Supplying sufficient oxygen to the engineered tissue is essential for survival and integration of transplanted cells. Unfortunately, the lack of vascularization of implanted tissues and inadequate removal of waste products prevents diffusion of oxygen into the interior of the scaffold. This makes the survival rate of the seeded cells very low, and in many instances, only the cells located near the surface of implant survive. Such limitations have led to a general conception that cell or tissue components may not be implanted in large volumes, as the delay in vasculogenesis often results in premature cell death due to the inadequate supply of oxygen and nutrients.

Oxygen diffusion is of crucial importance especially when building a clinically relevant sized tissue or organ. The distance that oxygen must diffuse between capillary lumen and a cell membrane is almost never more than 40 to 200 μm (Chow et al., 2001, Biophys J, 81(2):685-96; Chow et al., 2001, Biophys J, 81(2):675-84) whereas, in most clinical grafts, the distance for oxygen from the edge of the graft to the center of the graft is a minimum 5 mm, or approximately fifty times the normal diffusion distance (Muschler et al., 2004, J Bone Joint Surg Am, 86-A(7):1541-58). In this setting, diffusion is able to support only a limited number of transplanted cells, and this creates the center of the graft where oxygen tension is too low to support viable cells, resulting in central necrosis. This is a major reason why many cell transplantation methods work very well in small animals but fail in larger animals and humans. Currently, oxygen diffusion has been limiting the engineering of large functional tissue implants for human application.

Several methods have been developed to overcome this challenge. For example, strategies including the use of oxygen rich fluids such as perfluorocarbons and silicone oils (Radisic et al., 2006, Tissue Eng, 12(8):2077-91; Leung et al., 1997, J Chem Technol Biotechnology, 68:37-46), the use of angiogenic factors, such as vascular endothelial growth factors (VEGF) and endothelial cells, and cell-support matrices that permit enhanced diffusion across the entire implant (De Coppi et al., 2005, Tissue Eng, 11(7-8):1034-44; Kaigler et al., 2006, J Bone Miner Res, 21(5):735-44; Nomi et al., 2002, Mol Aspects Med, 23(6):463-83) have all been attempted. However, none of these strategies have been successful to date in achieving survival of a clinically applicable large tissue mass (Harrison et al., 2007, Biomaterials, 28(31):4628-34). Although these measures are designed to facilitate the delivery of oxygen, they are unable to reduce the oxygen demand of the cells.

One potential solution is to develop methods to maintain cell viability over a long-term by downregulating cellular metabolism until host vascularization is established. Adenosine, a purine nucleoside that functions as an energy transferring molecule, is known to be a key regulator in controlling metabolic activity (Boutilier™, 2001, J Exp Biol 204(Pt 18):3171-3181). It has been reported to increase in hypoxia-tolerant cells under hypoxic stress and reduce the ATP demands of the Na+/K+ ATPase, the dominant ATP consuming cellular process, especially under severe oxygen limitations (Buck, 2004, Comp Biochem Physiol B Biochem Mol Biol 139(3):401-414). This results in a decrease in ATP consumption and thus, oxygen demand.

One of the primary challenges of the cell-based tissue engineered constructs for achieving large sized and functional tissue implants for human applications is an inadequate supply of oxygen (Khademhosseini et al., 2006, Proc Natl Acad Sci USA 103(8):2480-2487). This is due to the delay of vasculogenesis and integration of vessels into the constructs after implantation. Insufficient oxygenation limits cellular energy metabolism resulting in hypoxic conditions within the scaffolds leading to cellular dysfunction and premature cell death. Ultimately, grafted cells do not survive and the constructs fail.

Ischemia is one of the biggest challenges in biomedical application whether it is associated with diseases, injuries, or medical treatment. Lack of blood supply causes various problems depending on the duration, location, and proportion of the ischemia damage. Severe ischemia leads to damages such as loss of limb, organ dysfunction, brain defect, and even lethal condition (Huang and Castillo, 2008, Radiographics 28(2):417-39). Particularly in cell-based tissue engineering purposes, it is critical to maintain a viable scaffold after implantation. However, it can be problematic when neovascularization into the construct is delayed. Then the cells in the scaffold have to survive under ischemic condition with limited oxygen until the host vasculature infiltration.

Considering the size of the constructs required for human application, it is known that cells can only survive within 200 μm from the outer boundaries of a construct in vitro (Malda et al., 2004, Biomaterials 25(26):5773-5780; Oh et al., 2009, Biomaterials 30(5):757-762; Radisic et al., 2006, Biotechnol Bioeng 93(2):332-343). As a consequence, constructs larger than 1 cm³ cannot rely solely on infiltration of host vasculature to remain viable in vivo as they typically become hypoxic and eventually necrotic (Davis et al., 2007, Ann Biomed Eng 35(8):1414-1424; Griffith et al., 2005, Tissue Eng 11(1-2):257-266; Ishaug-Riley et al., 1998, Biomaterials 19(15):1405-1412). Such necrosis occurs especially in the central region of the scaffold because oxygen tension becomes too low to support viable cells when the diffusion distance from the oxygen source at the periphery of the scaffold increases. The diffusion distance is estimated to have an inverse square relationship with the maximum concentration of cells. This is why constructs for large animals and humans often fails, while successful in smaller animals (Muschler et al, 2004, J Bone Joint Surg Am 86-A(7):1541-1558).

Given the negative effects of limited oxygen supply for most tissue-engineered constructs, a number of strategies have been explored to overcome this hindrance. These include the use of synthetic oxygen carriers such as perfluorocarbons (Iyer et al., 2007, Artif Cells Blood Substit Immobil Biotechnol 35(1):135-148; Tan et al., 2009, Tissue Eng Part A 15(9):2471-2480) and oxygen-generating biomaterials (Oh et al., 2009, Biomaterials 30(5):757-762; Pedraza et al., 2012, Proc Natl Acad Sci USA 109(11):4245-4250; Harrison et al., 2007, Biomaterials 28(31):4628-4634), and the incorporation of angiogenic factors such as vascular endothelial growth factor (VEGF) and endothelial cells to enhance neovascularization into the matrix (Grunewald et al., 2006, Cell 124(1):175-189; Kalka et al., 2000, Proc Natl Acad Sci USA 97(7):3422-3427). Another approach is the design of a microcirculation network within matrices that allows enhanced oxygen diffusion (Yang et al., 2002, J Biomed Mater Res 62(3):438-446). Facilitating oxygenation to the implants at the time of implantation is the common focus of these current strategies, however, none have for various reasons been successful to date in achieving survival of a clinically applicable large tissue mass (Oh et al., 2009, Biomaterials 30(5):757-762; Harrison et al., 2007, Biomaterials 28(31):4628-4634; Ness and Cushing, 2007, Arch Pathol Lab Med 131(5):734-741; Stowell et al., 2001, Transfusion 41(2):287-299; Kim and Greenburg, 2004, Artif Organs 28(9):813-828).

Thus, a need exists for a method of promoting cell survival under hypoxic conditions by exploiting this property of adenosine, or derivatives of adenosine, by prolonging the presence and/or activity of adenosine and its derivatives by inhibiting or reducing the manner in which a cell metabolizes such molecules. The present invention satisfies this need.

### SUMMARY OF THE INVENTION

The invention provides a method of increasing the viability of a cell under a hypoxic condition, comprising contacting the cell with an effective amount of adenosine or a derivative thereof to reduce the oxygen demand of the cell.

In one embodiment, the effective amount of adenosine or a derivative thereof downregulates the metabolic rate of the cell.

In one embodiment, contacting the cell with an effective amount of adenosine or a derivative thereof further results in a steady state of cellular metabolic activity.

In one embodiment, the cell resumes a normal proliferation rate when the adenosine or a derivative thereof is removed from the cell.

In one embodiment, the cell is a myoblast. In one embodiment, the cell is a murine myoblast. In one embodiment, the cell is a human myoblast.

The present invention also provides method of increasing cellular survival in a tissue-engineered construct during vasculogenesis, comprising administering an effective amount of adenosine or a derivative thereof to the cells in the tissue-engineered construct to downregulate the metabolic rate of the cells until host vascularization is established.

The present invention also provides a method of prolonging the survival of an implanted cell that is under a hypoxic condition in a host, comprising contacting the cell with an effective amount of adenosine or a derivative thereof to reduce the oxygen demand of the cell until host neovascularization is achieved.

In one embodiment, the effective amount of adenosine or a derivative thereof downregulates the metabolic rate of the cell.

In one embodiment, contacting the cell with an effective amount of adenosine or a derivative thereof further results in a steady state of cellular metabolic activity.

In one embodiment, the hypoxic cell resumes a normal proliferation rate when the effects of the adenosine or a derivative thereof are removed.

In one embodiment, the cell is a myoblast. In one embodiment, the cell is a murine myoblast. In one embodiment, the cell is a human myoblast.

The present invention also provides a method of increasing the viability of a cell under a hypoxic condition, comprising prolonging the availability of adenosine or a derivative thereof in the cell by contacting the cell with an effective amount of a purine metabolic enzyme inhibitor, such that the activity of the inhibited purine metabolic enzyme is reduced, and the prolonged availability of adenosine or a derivative thereof results in a reduction of the oxygen demand of the cell.

In one embodiment, the purine metabolic enzyme is adenosine deaminse.

In one embodiment, the purine metabolic enzyme inhibitor is selected from the group consisting of fludarabine phosphate, pentostatin, cladribine, coformycin, 2′-deoxycoformycin, erythro-9-(2-hydroxy-3-nonyl)adenine (EHNA), 9′-hydroxy-EHNA, 9′-chloro-EHNA, 9′-phthalimido-EHNA, 8′,9′-didehydro-EHNA, 1-deaza-EHNA, 3-deaza-EHNA, adechlorin, adecypenol, 1-deazaadenosine, 1-deaza-2′-deoxyadenosine, 3′-deoxy-1-deazaadenosine, 2′,3′-dideoxy-1-deazaadenosine, (2S,3R)-3-(6-amino-9H-purin-9-yl)-7-(o-tolyl)heptan-2-ol, erythro-9-(2-hydroxy-3-nonyl)-1,2,4-triazole, erythro-9-(2-hydroxy-3-nonyl)-1,2,4-triazole-3-carboxamide, kampherol, quercitin, 2-[4-[4,4-bis(4-fluorophenyl)butyl]piperazin-1-yl]-N-(2,6-dimethylphenyl)acetamide, dipyridamole, trazodone, or phenylbutazone.

In one embodiment, the purine metabolic enzyme inhibitor is cladribine.

In one embodiment, the cell resumes a normal proliferation rate when the purine metabolism enzyme inhibitor is removed from the cell.

The present invention also provides a method of increasing cellular survival in a tissue-engineered construct during vasculogenesis, comprising administering an effective amount of purine metabolism enzyme inhibitor to the cells in the tissue-engineered construct to prolong the availability of adenosine or a derivative thereof present in the cells, thereby downregulating the metabolic rate of the cells until host vascularization is established.

The present invention also provides method of prolonging the survival of an implanted cell that is under a hypoxic condition in a host, comprising contacting the cell with an effective amount of purine metabolism enzyme inhibitor to prolong the availability of adenosine or a derivative thereof, thereby reducing the oxygen demand of the implanted cell.

In one embodiment, the hypoxic cell resumes a normal proliferation rate when the effects of purine metabolism enzyme inhibitor are removed.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a chart depicting the targeted effect of adenosine on cellular activity with respect to time.

FIG. 2 is a chart depicting the effect of adenosine on C2C12 metabolic activity under either normoxic or hypoxic condition.

FIG. 3 is a chart depicting the effect of adenosine dose on cellular metabolic activity under hypoxic condition throughout.

FIG. 4 is a chart depicting the long term effect of adenosine on restoring cellular activity.

FIG. 5 is a schematic depicting the metabolic conversion of adenosine and a mechanism for inhibition of adenosine deaminase (ADA) by adenosine deaminase inhibitors.

FIG. 6 is a chart depicting the effect of adenosine derivatives on cell viability.

FIG. 7, comprising FIGS. 7A-7E, depicts the effect of adenosine and adenosine derivatives on cell viability. FIG. 7A is a chart depicting the comparative effects of adenosine and adenosine derivatives on cell viability when removed. Individual charts are depicted for adenosine (FIG. 7B), cladribine (FIG. 7C), fludaribine phosphate (FIG. 7D), and pentostatin (FIG. 7E).

FIG. 8 is a schematic diagram of exemplary mechanisms for prolonging cell survival under hypoxic conditions by manipulating two properties of adenosine on cells: 1) oxygen is required to produce ATP, the vital cellular energy source. Adenosine lowers the major ATP-consuming activities (Ion channel and protein synthesis). 2) Adenosine halts cellular growth by the induction of cell cycle arrest. Oxygen demand can be reduced by affecting either of these activities.

FIG. 9, comprising FIGS. 9A-9C, depicts the skin flaps on the backs of mice. FIG. 9A is a diagram depicting the predominant pattern of vascular anatomy in dorsal mouse skin and mechanism of skin flap model. FIG. 9B is a photograph depicting the design of ischemic flap model using silicone sheet and agarose gel as a vessel blocker and CDA carrier, respectively. FIG. 9C is a photograph depicting how CDA increases ischemic flap survival. Representative ischemic flaps showing better flap survival in CDA-treated group compared with one in control group.

FIG. 10 is a photograph depicting the histological analysis of skin flaps. H&E stains of the skin flaps harvested at 3 days showed delayed necrosis in the control group with better conservation of tissue architecture, thickness of the skin and epidermis height (black arrows).

FIG. 11, comprising FIGS. 11A-11B, depicts muscle function after the compression injury. FIG. 11A is a chart depicting the determination of muscle function 3 days after compression injury. A greater increase in muscle force was observed in the CDA-treated group. FIG. 11B is a chart depicting percent recovery of muscle function. The muscle function of both groups recovered with respect to time, however, the CDA-treated group showed a significant increase in percent recovery at both 3 and 7 days. * Student t-test analysis at P<0.05, n=6 per each group.

FIG. 12, comprising FIGS. 12A through 12C is a series of images demonstrating that adenosine enhances C2C12 survival under 0.1% hypoxic conditions and preserves cell function. FIG. 12A demonstrates that 5 mM adenosine treated hypoxic cells survived hypoxic stress and regained normal growth when transferred to normoxic conditions without further supply of adenosine, whereas the control cells did not survive by 11 (n=4). FIG. 12B shows representative fluorescent images of live C2C12 cells stained with calcein AM which demonstrates a consistent cell population throughout the hypoxic duration and is increased under normoxic conditions only in the adenosine treated group (scale bars=200 μm). FIG. 12C shows representative images of myotubes formed in normoxic and hypoxia-survived C2C12 cells when cultured in 2% horse serum-containing differentiating medium for 6 days under normoxic conditions. Cells were immonostained with MF-20 (myosin heavy chain) and DAPI (nuclei), and stained with Giemsa (scale bars=200 μm).

FIG. 13, comprising FIGS. 13A and 13B, is a series of images showing the effect of adenosine on metabolic activity of C2C12 cells. FIG. 13A shows MTS metabolic activity per cell (n=4) and (FIG. 13B) the cumulative metabolic activity per cell, represented by the area under the curve in (FIG. 13A), at specified hypoxic duration. Metabolic activity was suppressed initially only when treated with 5 mM adenosine (n=4, Student t-test, *P<0.05 vs. controls and 0.05 mM group). This downregulated metabolic activity was sustained at a fairly consistent level throughout the hypoxic duration, and was restored to a normal level when adenosine treated hypoxic cells were transferred to normoxic conditions.

FIG. 14 is an image demonstrating the effect of adenosine on intracellular ATP level of C2C12 cells under hypoxia. Intracellular ATP level of the control and the 5 mM adenosine treated cells were expressed as a percentage of the total level measured in the normoxic cells. ATP level of the controls was 19% at day 3, continued to decline until no ATP was detected at day 11. A consistently higher ATP level was observed in the adenosine treated cells throughout the hypoxic duration (n=4, Student t-test, *P<0.05), still maintaining 18% of ATP at day 11.

FIG. 15 is an image demonstrating the effect of adenosine concentrations on C2C12 survival. Cells grown under all concentrations of adenosine survived 0.1% hypoxia and re-proliferated in normoxic conditions except for 0.05 mM. The effect of concentrations was reflected on advancing the onset of re-proliferation after transfer to normoxic conditions (n=4).

FIG. 16, comprising FIGS. 16A and 16B, is a series of images showing the effects of adenosine on metabolic activity. FIG. 16A shows long-term effect of adenosine on C2C12 cell survival. MTS metabolic activity of the cells was suppressed for 22 days while under the effect of 5 mM adenosine in 0.1% hypoxic conditions, and still re-proliferated when its effect was removed (n=4). FIG. 16B shows the effect of adenosine on human primary cells. Such effect was also observed on hMPCs when subjected to hypoxic conditions for 7 days (n=4).

FIG. 17, comprising FIGS. 17A through 17C, is a series of images demonstrating that adenosine protects hypoxic tissues. FIG. 17A shows representative H&E images of soleus muscle tissues after 10 days of culture in hypoxic conditions (scale bar=100 μm). The tissues cultured without adenosine show more damage than that of tissues treated with 5 mM adenosine, indicated by degenerated myofibers and loss of connective tissues. FIG. 17B shows representative fluorescent images of tissues stained with EthD-1 (dead cells) and DAPI (nuclei) (scale bar=100 μm). FIG. 17C shows quantitative analysis on number of dead cells expressed as a percentage of total number of cells stained with DAPI based on images in FIG. 17B. The 5 mM adenosine treated tissues showed a significantly less number of dead cells compared to the controls (n=3, Student t-test, *P<0.05).

FIG. 18 is an image showing percent recovery of muscle function after compression injury. The muscle function of both no- and CDA-groups recovered with respect to time, however, the CDA-treated group showed a significant increase in percent recovery up to day 11 (n=6, Student t-test, *P<0.05).

FIG. 19, comprising FIGS. 19A through 19B, is a series of images demonstrating cell survival using adenosine under hypoxic conditions. FIG. 19A shows a graph depicting how adenosine enhances C2C2 cell survival under hypoxia. C2C12 cells (2,500 cells per well) were cultured for 11 days under 0.1% hypoxic conditions followed by normoxic conditions without further supply of adenosine. The number of cells was assessed via dsDNA content. All the experimental groups with various concentrations of adenosine (1, 2 and 5 mM) survived hypoxic stress (n=4). After a transfer to normoxic conditions, these cells re-proliferated with a growth rate comparable to that of normoxic cells, whereas the control cells did not re-proliferate. The onset of re-proliferation was concentration-dependent. An earlier onset was observed with an increase of concentration. The normoxic, 2 mM and 5 mM adenosine-treated cells showed their number declined after becoming fully confluent at time points immediately following their highest numbers. Those declined curves are not shown. FIG. 19B shows representative fluorescent images of live C2C12 cells stained with calcein AM (green), without (Upper) and with 5 mM adenosine (Lower). These images provide support that a consistent cell population throughout the hypoxic duration and its increase under normoxic conditions was shown only in the adenosine-treated group (scale bars=200 μm) (also see FIG. 19A).

FIG. 20 shows representative images of myotubes formed only in hypoxia-survived C2C12 cells in the presence adenosine. C2C12 cells underwent identical testing conditions as described in FIG. 19 except that these cells were cultured in the complete growth medium for 3 days after transfer to normoxic conditions followed by 2% horse serum-containing differentiating medium for 6 days. C2C12 cells were immonostained (Upper) with MF-20 (myosin heavy chain, red) and DAPI (nuclei, blue), and stained with Giemsa (Lower) (scale bars=200 μm).

FIG. 21 shows a graph depicting the effect of adenosine on metabolic activity of a single C2C12 cell assessed via MTS metabolic activity and dsDNA content (n=4). When treated with 5 mM adenosine under 0.1% hypoxic conditions, metabolic activity was shifted to the right of that of the control group, resulting in its downregulated states approximately for the first 5 days. When these cells were transferred to normoxic conditions, metabolic activity was restored to a normal level that was measured in normoxic cells. A normal level is shown only up to day 3 because a decline in cell number was observed in the following time points.

FIG. 22 is a graph depicting the effect of adenosine on intracellular ATP level of a single C2C12 cell during the 0.1% hypoxic phase. Intracellular ATP of hypoxic cells was expressed as a percentage of that measured in normoxic cells at day 0. With only 19% remained at day 3, ATP of the control group continued to decline until no ATP was detected at day 11. Throughout the hypoxic duration, a higher ATP level was observed in the cells treated with 5 mM adenosine, still maintaining 18% of ATP by day 11 (n=4, Student t-test, *P<0.05).

FIG. 23, comprising FIGS. 23A through 23C, is a series of images demonstrating that adenosine protects hypoxic tissues. FIG. 23A shows representative H&E images of soleus muscle tissues after 10 days of culture in hypoxic conditions (scale bar=100 μm). The tissues cultured without adenosine show more damage than that of tissues treated with 5 mM adenosine, indicated by degenerated myofibers and loss of connective tissues. FIG. 23B shows representative fluorescent images of tissues stained with EthD-1 (dead cells, red) and DAPI (nuclei, blue) (scale bar=200 μm). FIG. 23C shows a graph depicting quantitative analysis on the number of dead cells expressed as a percentage of total number of cells stained with DAPI based on the images of FIG. 23B. The 5 mM adenosine treated tissues showed a significantly fewer dead cells compared to the controls (n=3, ANOVA with Tukey's post hoc test, *P<0.01).

FIG. 24, comprising FIGS. 24A through 24B, is a series of images demonstrating that adenosine was able to maintain cell population significantly lower than the no treatment control when used in the absence of an ADA inhibitor. FIG. 24A shows a series of graphs demonstrating that both adenosine and cladribine maintained a cell population significantly lower than the no treatment control when used in the absence of an ADA inhibitor. FIG. 24B is a series of fluorescent images demonstrating the results depicted in FIG. 24A. From the left column, images show cells with no treatment (control), adenosine-treated cells, and cladribine-treated cells. From the top row, images show cells day 1 after drug treatment, day 7, and day 11 when drugs were removed. No treatment control showed mostly dead cells at day 11 indicating the cells were not able to survive the hypoxic condition. Adenosine-treated groups showed significant proliferation at day 7 and at day 11, while cladribine-treated cells showed minimal cell population at day 7 and proliferated cells at day 11.

FIG. 25, comprising FIGS. 25A through 25B, is a series of images demonstrating the effect of adenosine and an ADA inhibitor, cladribine, on cell survival in hypoxia. FIG. 25A shows the effect of adenosine and an ADA inhibitor, cladribine, on cell survival in hypoxia. To simulate adenosine degradation in physiological condition, ADA was treated with each ADA inhibitor. The cells were treated with each ADA inhibitor in combination with ADA, and then incubated for 7 days under hypoxic condition (unshaded area). At day 7, the cells were moved to normoxic condition (shaded area) and the media was changed with fresh completed media with or without drugs. No treatment control was represented as black X, adenosine- and cladribine-treated cells were represented with red cubic and blue rhombus, respectively. Open heads represent drug-free cells and filled heads represent cells with drugs and ADA, after day 7. Adenosine failed to maintain cell population showing similar proliferation with no treatment control and failed in long-term cell survival. Cladribine-treated C2C12 cells maintained cell population under the influence of drug and regained their proliferation when the drugs and hypoxia were removed. Two-way ANOVA with Bonferroni posttest was used for statistical analysis (*, p<0.05; ***, p<0.001). FIG. 25B is a series of fluorescent images demonstrating the results depicted in FIG. 25A. From the left column, images show cells with no treatment (control), adenosine-treated cells, and cladribine-treated cells. From the top row, images show cells day 1 after drug treatment, day 7, and day 11 when drugs were removed. No treatment control showed mostly dead cells at day 11 indicating the cells were not able to survive the hypoxic condition. Adenosine-treated groups showed significant proliferation at day 7 and very low number of cells at day 11, whereas cladribine-treated cells showed minimal cell population at day 7 and proliferated cells at day 11.

FIG. 26, comprising FIGS. 26A through 26B, is a series of images demonstrating the effect of an ADA inhibitor (cladribine) and adenosine on cellular metabolism in hypoxia in the absence of an ADA. FIG. 26A shows a series of graphs demonstrating the effect of an ADA inhibitor (cladribine) and adenosine on cellular metabolism in hypoxia Shaded area indicates that the cells were in normoxic incubator. Both adenosine- and cladribine-treated C2C12 cells kept metabolism to a minimum under the influence of drug and regained their normal metabolism when the drugs and hypoxia were removed. FIG. 26B shows a graph demonstrating cellular metabolism per cell under hypoxic condition. Cladribine showed lower cellular metabolism when compared to adenosine. Two-way ANOVA with Bonferroni posttest was used for statistical analysis. (***, p<0.001).

FIG. 27, comprising FIGS. 27A through 27B, is a series of images demonstrating the effect of an ADA inhibitor (cladribine) and adenosine on cellular metabolism in hypoxia. FIG. 27A shows a series of graphs demonstrating the effect of an ADA inhibitor (cladribine) and adenosine on cellular metabolism in hypoxia Shaded area indicates that the cells were in normoxic incubator. Adenosine failed to maintain cell metabolism in presence of ADA. Cladribine-treated C2C12 cells kept metabolism to a minimum under the influence of drug and regained their normal metabolism when the drugs and hypoxia were removed. FIG. 27B shows a graph demonstrating cellular metabolism per cell under hypoxic condition. Cladribine showed significantly lower cellular metabolism when compared to adenosine. Two-way ANOVA with Bonferroni posttest was used for statistical analysis. (***, p<0.001).

FIG. 28, comprising FIG. 28A through FIG. 28D, is a series of images demonstrating in vitro ischemic TA muscle incubation. FIG. 28A shows H&E staining result demonstrating the most preserved muscle structure in cladribine-injected TA when compared to blank- and adenosine-injected muscles. FIG. 28B shows images demonstrating that ADA inhibitor decreased or delayed apoptotic and necrotic cell death under hypoxic environment showing significantly increased number of live nuclei in TUNEL staining compared to blank- and adenosine-injected muscles. FIG. 28C shows a graph demonstrating total areas of muscles in the pictures were calculated using ImagePro 6.3 software. ADA inhibitor-injected muscles showed significantly larger area of remaining muscle when compared to blank- and adenosine-injected muscles. (One-way ANOVA with Bonferroni posttests, *, p<0.05; **, p<0.01; ***, p<0.001, n=4). FIG. 28D shows a graph demonstrating that ADA inhibitor decreased or delayed apoptotic and necrotic cell death under hypoxic environment showing significantly increased number of live nuclei in TUNEL staining compared to blank- and adenosine-injected muscles.

FIG. 29, comprising FIGS. 29A through 29C, is a series of images demonstrating results of a skin flap model. FIG. 29A shows an image demonstrating the design of ischemic flap model using silicone sheet and 2% low-gelling agarose gel as a vessel blocker and CDA carrier, respectively. FIG. 29B shows a series of images demonstrating that CDA increases ischemic flap survival. Representative ischemic flaps showing better flap survival in CDA-treated group compared with one in control group. FIG. 29C shows a series of images demonstrating histological analysis: H&E stains of the skin flaps harvested at 3 days showed delayed necrosis in the control group with better conservation of tissue architecture, thickness of the skin and epidermis height (black arrows).

FIG. 30, comprising FIGS. 30A through 30C, is a series of images demonstrating the muscle function after compression injury. FIG. 30A shows a graph demonstrating the determination of muscle function 7 days after compression injury. A greater increase in muscle force was observed in the CDA-treated group. FIG. 30B shows a graph demonstrating the percent recovery of muscle function. The muscle function of both groups recovered with respect to time as the injury heals itself in this animal model with respect to time, however, the CDA-treated group showed a significant increase in percent recovery at both 3, 7 and 11 days (Student t-test, * P<0.05, n=6). FIG. 30C shows a series of images demonstrating that qualitative analyses on H&E stains and immunostainings against vWF of cross-sections of the saline CDA group still shows the increased diameter and space between the individual muscle fibers, and less new forming vessels respectively.

### DETAILED DESCRIPTION OF THE INVENTION

The present invention is directed towards a system and method of maintaining and/or increasing cell viability by downregulating cellular metabolic rate under hypoxic conditions. This concept also represents a novel method for increasing cellular survival in tissue-engineered constructs during vasculogenesis, whereby cell viability is increased by downregulating cellular metabolic rate until host vascularization is established. The present invention also relates to a system and method of prolonging the survival of implanted cells that are under hypoxic condition until host neovascularization is achieved. These systems and methods are achieved by contacting the cells or systems with adenosine, an adenosine derivative, a purine metabolism enzyme inhibitor, or any combination thereof.

### DEFINITIONS

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

As used herein, the term “treatment” or “treating” is defined as the application or administration of a therapeutic agent, i.e., a compound, such as adenosine, useful within the invention (alone or in combination with another agent), to a subject, or application or administration of a therapeutic agent to an isolated tissue or cell either engineered or from a subject (e.g., for diagnosis or ex vivo applications), with the purpose to cure, heal, alleviate, relieve, alter, remedy, ameliorate, improve or affect the condition being treated.

As used herein, the term “patient” or “subject” refers to a human or a non-human animal. Non-human animals include, for example, livestock and pets, such as ovine, bovine, porcine, canine, feline and murine mammals. Preferably, the patient or subject is human.

As used herein, the terms “effective amount,” “pharmaceutically effective amount” and “therapeutically effective amount” refer to a non-toxic but sufficient amount of an agent to provide the desired biological result. That result can be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “derivative” refers to a small molecule that differs in structure from the reference molecule, but retains the essential properties of the reference molecule. A derivative may change its interaction with certain other molecules relative to the reference molecule. A derivative molecule may also include a salt, an adduct, or other variant of the reference molecule.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Methods of the Invention

As demonstrated herein, cells (such as murine myoblasts and human myoblasts, for example) under hypoxic condition maintain a steady state of metabolic activity when treated with adenosine. Hypoxic cells not treated with adenosine proceed to die. Hypoxic cells can resume their normal proliferation rate when the effects of adenosine are removed. Thus, the present invention is directed towards a system and method of maintaining and/or increasing cell viability by down-regulating cellular metabolic rate under hypoxic conditions.

In one embodiment of the present invention, the method is performed by applying adenosine and/or any derivative thereof to cells under hypoxic conditions and prolonging cell survival by decreasing the metabolic activity to a steady hypometabolic state, thus reducing O₂ demand. In another embodiment, the method is performed by applying at least one purine metabolism enzyme inhibitor to cells under hypoxic conditions and prolonging cell survival by decreasing the metabolic activity to a steady hypometabolic state, thus reducing O₂ demand.

In one embodiment, the invention provides a method of using adenosine to promote long-term effects of on cell survival. In some instances, long-term is at least 10 days, in one aspect long-term is at least 15 days, in one aspect long-term is at least 20 days, in one aspect long-term is at least 25 days, in one aspect long-term is at least 30 days, in one aspect long-term is at least 35, in one aspect long-term is at least 40 days, in one aspect long-term is at least 45, in one aspect long-term is at least 50.

In another embodiment, the present invention includes methods of reducing the rate of adenosine metabolism in a cell to prolong the availability of functional adenosine and/or derivative thereof present in the cell. Adenosine is known to be converted to other compounds by purine metabolism enzymes, whereby conversion results in deactivation of the desired function of adenosine with respect to at least prolonging cell survival by decreasing the metabolic activity to a steady hypometabolic state. A non-limiting example of a purine metabolism enzyme is adenosine deaminase (ADA). ADA irreversibly converts adenosine to inosine through replacement of the C(6) amino group with a ketone, resulting in regain of oxygen demand because less adenosine is present in the system and inosine does not actively downregulate cellular metabolic rate. Therefore ADA interferes with the desired function of adenosine with respect to prolonging cell survival by decreasing the metabolic activity to a steady hypometabolic state.

Accordingly, one embodiment of the invention includes inhibiting or reducing the activity of at least one purine metabolism enzyme, thereby preventing adenosine or derivatives thereof from being converted into other compounds, and retaining the amount of adenosine or derivatives thereof present in the system. In another embodiment, the inhibited purine metabolism enzyme is adenosine deaminase. In another embodiment, the method is performed by applying an adenosine deaminase inhibitor, thereby preventing adenosine from being converted into inosine, resulting in prolonged availability of adenosine or derivatives thereof present in the system. In another embodiment, the adenosine deaminase inhibitor is cladribine (CDA). In another embodiment, the adenosine deaminase inhibitor is applied to cells under hypoxic conditions in place of adenosine to prolong cell survival by down-regulating the metabolic activity to a steady hypometabolic state, thus reducing O₂ demand. For example, the adenosine deaminase inhibitor, cladribine, can be used in replacement of adenosine to inhibit ADA activity and to maintain the effect of adenosine, resulting in long-term cell survival. Due to the minor structural difference of cladribine as compared to adenosine, cladribine is not converted by ADA and therefore maintains its activity of downregulating the metabolic activity of cells.

In another aspect of the present invention, the effects of adenosine and/or derivatives thereof, or inhibitors of purine metabolism enzymes on either inhibition of purine metabolism enzymes, cellular metabolic activity or proliferation are dose dependent. In one embodiment of the present invention, the effective concentration of adenosine and/or derivatives thereof, or inhibitors of purine metabolism enzymes to be administered to the cells is greater than about 0.01 μM. In another embodiment, the effective concentration of the administered molecule or compound is between about 0.01 μM to about 100 mM and any and all whole or partial increments therebetween, including about 0.1 μM, about 1 μM, about 0.01 mM, about 0.1 mM, about 1 mM, about 10 mM, and about 100 mM. As the dose of the administered molecule or compound increases from 0.01 μM to 100 mM, an escalation of steady hypometabolic state can be maintained under hypoxic conditions, such that the cells are able to resume their normal metabolic activity after a period of time, such as after 7 days.

In one embodiment, the effective concentration of adenosine is 1 mM. In another embodiment, the effective concentration of adenosine is 2 mM. In a particular embodiment, the effective concentration of adenosine is 5 mM. In one embodiment, the effective concentration of cladribine is 5 mM. In another embodiment, the effective concentration of cladribine is 30 mM.

The regimen of administration may affect what constitutes an effective amount. Therapeutic formulations may be administered to the cells either prior to or after a determination of cellular hypoxic levels. Further, several divided dosages, as well as staggered dosages may be administered daily or sequentially, or the dose may be continuously infused, or may be a bolus injection. Further, the dosages of any therapeutic formulations may be proportionally increased or decreased as indicated by the exigencies of the therapeutic or prophylactic situation.

Administration of adenosine and/or derivatives thereof, or inhibitors of purine metabolism enzymes to a cell may be carried out using known procedures, at dosages and for periods of time effective to inhibit purine metabolism enzymes and/or to treat hypoxic levels in the cell. An effective amount of adenosine and/or derivatives thereof, or inhibitors of purine metabolism enzymes necessary to achieve a therapeutic effect may vary according to factors such as the state of the disease or disorder in the cell or subject, and the ability of adenosine and/or derivatives thereof, or inhibitors of purine metabolism enzymes to treat hypoxic levels in the cell. Dosage regimens may be adjusted to provide the optimum therapeutic response. For example, several divided doses may be administered daily or the dose may be proportionally reduced as indicated by the exigencies of the therapeutic situation. One of ordinary skill in the art would be able to study the relevant factors and make the determination regarding the effective amount of adenosine and/or derivatives thereof, or inhibitors of purine metabolism enzymes without undue experimentation.

Actual dosage levels of adenosine and/or derivatives thereof, or inhibitors of purine metabolism enzymes may be varied so as to obtain an amount of inhibitors of purine metabolism enzymes that is effective to achieve the desired therapeutic response for a particular cell, composition, and mode of administration, without being toxic to the cell or subject.

In one embodiment, the compositions of the invention are formulated using one or more pharmaceutically acceptable excipients or carriers. In one embodiment, the pharmaceutical compositions of the invention comprise a therapeutically effective amount of adenosine and/or derivatives thereof, or inhibitors of purine metabolism enzymes and a pharmaceutically acceptable carrier.

Formulations including adenosine and/or derivatives thereof, or inhibitors of purine metabolism enzymes may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The present invention is also directed towards a system and method of increasing cellular survival in tissue-engineered constructs during vasculogenesis. In one embodiment, the method is performed by applying adenosine and/or derivatives thereof to cells to downregulate cellular metabolic rate until host vascularization is established. In another embodiment, the method is performed by inhibiting the activity of at least one purine metabolism enzyme, thereby preventing or reducing the rate of adenosine and/or derivatives thereof from being converted into other compounds, resulting in prolonging the presence or availability of adenosine and/or derivatives thereof in the system. In another embodiment, the method is performed by applying at least one purine metabolism enzyme inhibitor to cells to down-regulate cellular metabolic rate until host vascularization is established.

The present invention further relates to a system and method of prolonging the survival of implanted cells that are under hypoxic condition until host neovascularization is achieved. In one embodiment, the method is performed by adding adenosine and/or derivatives thereof to cells to downregulate cellular metabolic rate. In another embodiment, the method is performed by inhibiting the activity of at least one enzyme involved in purine metabolism, thereby preventing or reducing the rate of adenosine and/or derivatives thereof from being converted into other compounds, resulting in prolonging the presence or availability of adenosine and/or derivatives thereof in the system. In another embodiment, the method is performed by adding at least one purine metabolism enzyme inhibitor to cells to downregulate cellular metabolic rate.

The present invention further relates to a system and method of increasing the viability of a cell under a hypoxic condition, consisting of applying adenosine and at least one purine metabolism enzyme inhibitor to cells to prolong the availability of adenosine and/or derivatives thereof in the cell, resulting in a reduction of the oxygen demand of the cell, thereby prolonging cell survival.

The methods of the present invention are markedly different than existing methods, in that the present invention works by lowering the oxygen demand of the cells by downregulating their metabolic rate to a hypometabolic steady state, instead of focusing on preventing the extreme hypoxia that immediately follows implantation of cells to the scaffold by facilitating the delivery of oxygen.

The present invention can be incorporated into the use of biomaterials for medical implants, devices, scaffolds, etc. The present invention may also be used to supplement cell culture media for controlled cell growth. Additionally, the present invention may be used with various formulations administered as an injection, ointment, dressing or spray, for example, to treat ischemia and trauma.

As contemplated herein, the present invention may be used in conjunction with the engineering of clinically relevant tissues for functional recovery, tissue and organ salvage due to trauma and ischemia of various tissues and organs, organ transplantation and reconstructive procedures involving tissue flap and grafts (intra and post-operative supplements). For example, the present invention is vital for extending the viability of larger engineered constructs seeded with higher densities of cells in vivo. With vascularization of tissue scaffolds estimated at 0.5-1 mm/day in tissue, maintaining cell viability in the middle of a tissue scaffold for 10 days permits the use of centimeter sized tissue scaffolds (Cao et al., 2006, Biomaterials, 27(14):2854-64).

In one embodiment, the method is performed by contacting the clinically relevant tissue with adenosine and/or derivatives thereof to promote cell viability and by downregulating cellular metabolic rate tissue until host neovascularization is achieved. In another embodiment, the method is performed by inhibiting the activity of at least one purine metabolism enzyme, thereby preventing adenosine and/or derivatives thereof from being converted into other compounds, resulting in more adenosine and/or derivatives thereof present in the system. In another embodiment, the method is performed by contacting the clinically relevant tissue with an inhibitor of a purine metabolism enzyme to promote cell viability by downregulating cellular metabolic rate tissue until host neovascularization is achieved. In another embodiment, the clinically relevant tissue is a tissue flap. In another embodiment, the inhibitor of a purine metabolism enzyme is cladribine. In another embodiment, the method is performed by contacting a tissue flap with cladribine to decrease tissue necrosis and/or increase muscle function.

In instances where severe oxygen limitation is present, cell death occurs when ATP production fails to meet the energetic maintenance demands of ionic and osmotic equilibrium. The decline of high energy phosphates level leads to a failure of ion-motive ATPases, followed by membrane depolarization, which leads to uncontrolled cellular swelling and, ultimately, to cell necrosis. Ion-motive ATPase is one of the dominant energy-consuming processes of cells at standard metabolic rate (19-28%). Under cellular stress, priority of energy consumption even shifts from protein synthesis to more critical cell function involved in osmotic and ionic homeostasis (20-80%). One of the mechanisms is the passive ion channel arrest, resulting in decreases in membrane permeability (“ion channel arrest”) that dramatically reduce the energetic costs of ion-balancing ATPases.

Addition of Adenosine, Adenosine Derivatives and/or Purine Metabolism Enzyme Inhibitors

Adenosine, a nucleoside known for its function as an energy transferring molecule, is also known to function as a modulator of hypoxia-induced ion-channel arrest, which eventually leads to lowering of ATP consumption, and thus oxygen demand. Moreover, it is also known to stimulate angiogenesis. Thus, the present invention provides for the application of adenosine and/or derivatives thereof to cell-seeded scaffolds under the hypoxic condition to improve cell survival rate via the downregulation of metabolic rate, and thus lowering oxygen demand and consumption. As contemplated herein, adenosine derivatives, as well as agonists to adenosine, may also be used in a similar manner to lower the oxygen demand of cells and tissues. Similar results are observed when adenosine and/or derivatives thereof are injected into muscle tissue, demonstrating the preservation of cellular viability and tissue architecture. Adenosine and/or derivatives thereof may be administered alone as a composition or within a formulation, as would be understood by those skilled in the art.

In another embodiment, the present invention includes methods of applying purine metabolic enzyme inhibitors to cell-seeded scaffolds under the hypoxic condition to improve cell survival rate via the downregulation of metabolic rate, and thus lowering oxygen demand and consumption. In one embodiment, the purine metabolic enzyme inhibitor is cladribine. Cladribine, when used as an adenosine replacement, inhibits ADA activity and maintains the same effect of down-regulating the metabolic rate as adenosine, thereby lowering oxygen demand and consumption, resulting in long-term cell survival. Due to the minor structural difference, cladribine is not converted to a different compound by ADA and therefore maintains its activity of downregulating the metabolic activity of cells. Purine metabolic enzyme inhibitors may be administered alone as a composition or within a formulation, as would be understood by those skilled in the art.

Compounds useful within the methods of the invention include any inhibitor of purine metabolism enzymes known in the art, including their salts, hydrates, solvates, clathrates, prodrugs, and analogs thereof. Therefore, the disclosures of all prior art related to the inhibition of purine metabolism enzymes are included herein in their entireties. Non-limiting examples of adenosine deaminase inhibitors include: fludarabine phosphate, pentostatin, cladribine, coformycin, 2′-deoxycoformycin, erythro-9-(2-hydroxy-3-nonyl)adenine (EHNA), 9′-hydroxy-EHNA, 9′-chloro-EHNA, 9′-phthalimido-EHNA, 8′,9′-didehydro-EHNA, 1-deaza-EHNA, 3-deaza-EHNA, adechlorin, adecypenol, 1-deazaadenosine, 1-deaza-2′-deoxyadenosine, 3′-deoxy-1-deazaadenosine, 2′,3′-dideoxy-1-deazaadenosine, (2S,3R)-3-(6-amino-9H-purin-9-yl)-7-(o-tolyl)heptan-2-ol, erythro-9-(2-hydroxy-3-nonyl)-1,2,4-triazole, erythro-9-(2-hydroxy-3-nonyl)-1,2,4-triazole-3-carboxamide, kaempferol, quercetin, lidoflazine, dipyridamole, trazodone, or phenylbutazone.

The methods described herein may also comprise the administration of one or more other therapeutic agents. In one embodiment, the methods described herein comprise the administration of adenosine in combination with inhibitors of enzymes involved in purine metabolism.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Example 1

### Effect of Adenosine and its Characterization on Cellular Activity

The following studies were performed to evaluate whether: 1) hypoxic cells not treated with adenosine result in necrosis; 2) cells under hypoxic condition maintain a steady state of metabolic activity when treated with adenosine; and 3) hypoxic cells resume their normal proliferation rate when the effects of adenosine is removed.

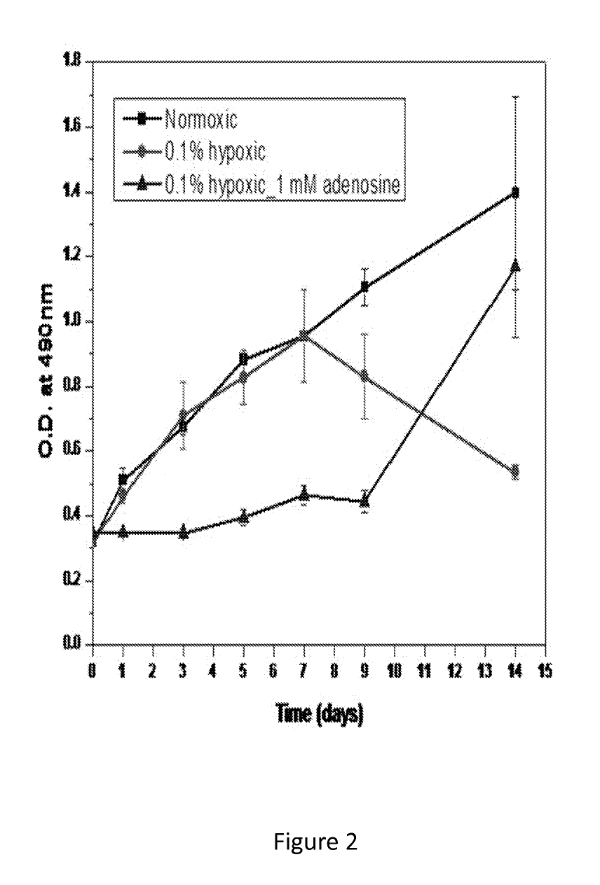

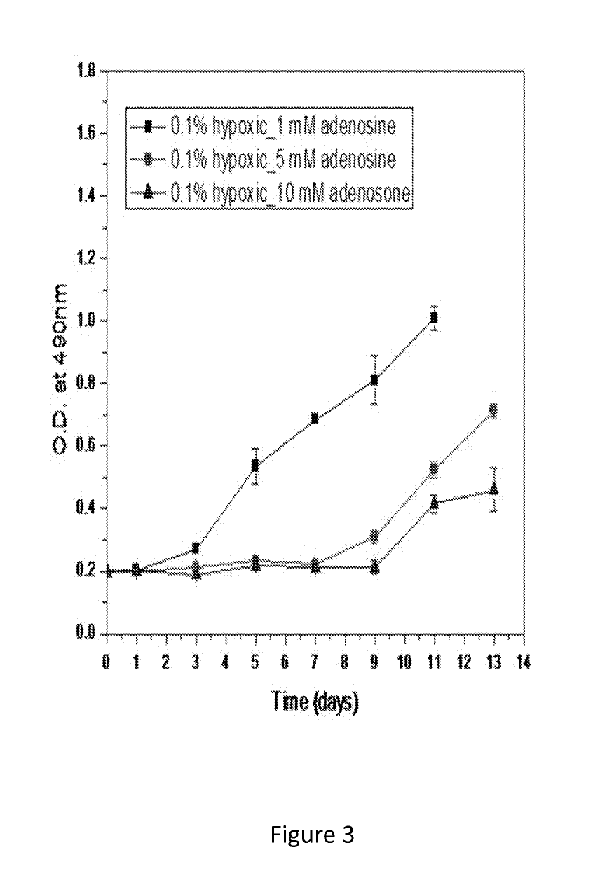

500 μL of a cell suspension (C2C12 cells, murine myoblasts) in high glucose Dulbecco's modified Eagle's medium (DMEM, Gibco) containing 10% FBS, 500 U/mL penicillin and 500 μg/mL streptomycin was placed in each well of a 48-well culture plate at a density of 1052 (FIG. 2) and 2105 (FIG. 3) cells/cm². Cells were incubated for 24 hr in normoxic conditions (21% O₂, 37° C.) prior to placement in a hypoxic chamber. At day 0, the plates designated as the hypoxic group were transferred to the hypoxic chamber (0.1% O₂). A group with no adenosine was placed under hypoxia for up to 13 days to demonstrate eventual cell death. Another group receiving daily doses of adenosine (0, 0.025, 0.25, 1, 5, and 1 mM adenosine, achieved by serial dilution) was incubated for up to 7 days under hypoxia and then placed back into normoxic conditions without additional supply of adenosine. Adenosine was refreshed by the daily exchange of media in which adenosine was completely dissolved. Media to be used under hypoxia was placed in the hypoxic chamber 24 hr prior to use for deoxygenation down to 2% O₂. The metabolic activity of viable cells at each pre-determined time point was assessed using an MTS assay, which measures mitochondrial activity of cells.

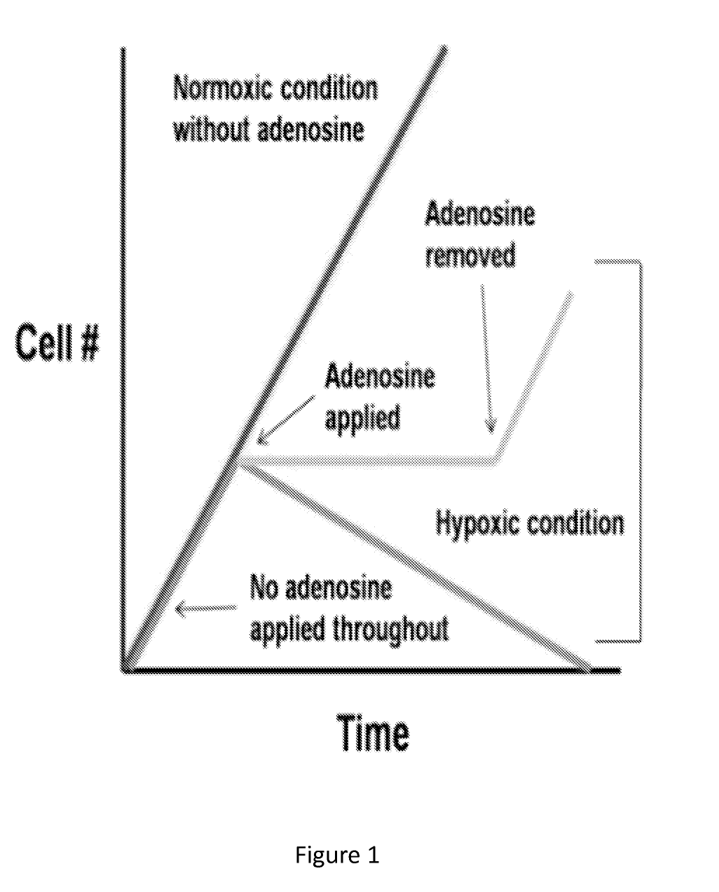

As depicted in FIG. 1, hypothetical clinical settings are simulated where seeded cells not treated with adenosine prior to host neovascularization result in cell death after implantation, but ones under the effect of adenosine maintains its viability for an extended period. Importantly, cells have to restore their normal metabolic activity and proliferation rate upon neovascularization, as the long-term maintenance of a suppressed state might not be clinically meaningful. Thus, the time period for the transition process from the hypometabolic phase to take place, and the recovered proliferation rate once the effect of adenosine is removed, are significant parameters to be evaluated.

Adenosine was used with C2C12 at passage 17, the murine myoblast cell lines, because of their relatively high metabolic activity and proliferation rate. This study demonstrated that the metabolic activity of cells grown in normoxic conditions increased linearly with respect to time (FIG. 2). Hypoxic cells not treated with adenosine showed a similar pattern of increasing metabolic activity up to 7 days under hypoxia, but this resulted in eventual decrease in cellular activity after day 7. Based on the microscopic observations of these cell stained with Giemsa, it was not seen that whole population of cells completely reached the state of necrosis up to the latest time point tested, as a few populations of attached cells were still observed. It is expected that those remaining cells eventually die. However, the cells that were supplied with adenosine under the hypoxic conditions maintained a steady state of cellular activity, and these cells resumed their normal metabolic activity two days after adenosine was removed at day 7.

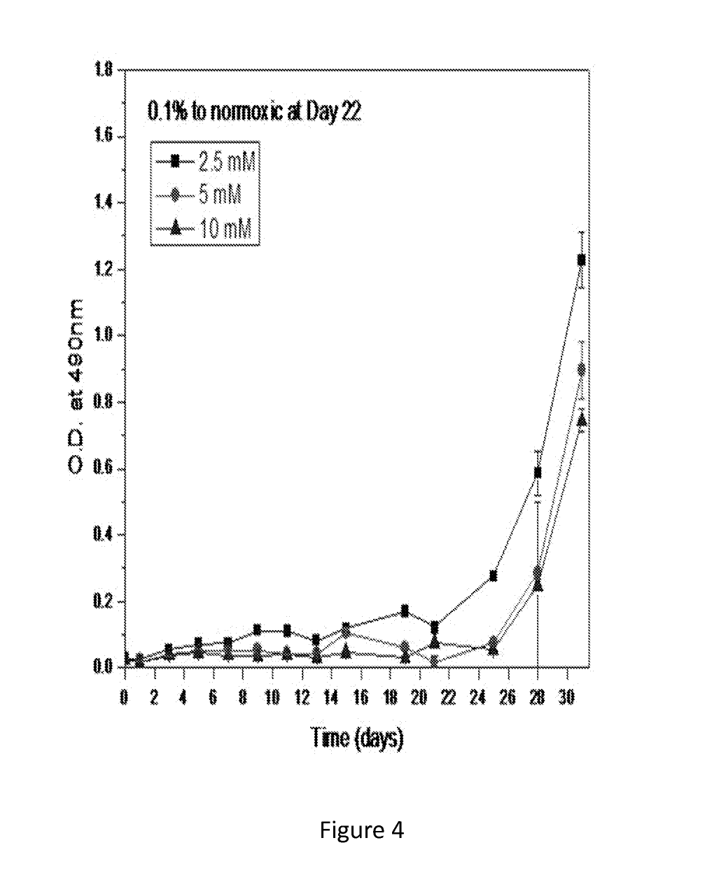

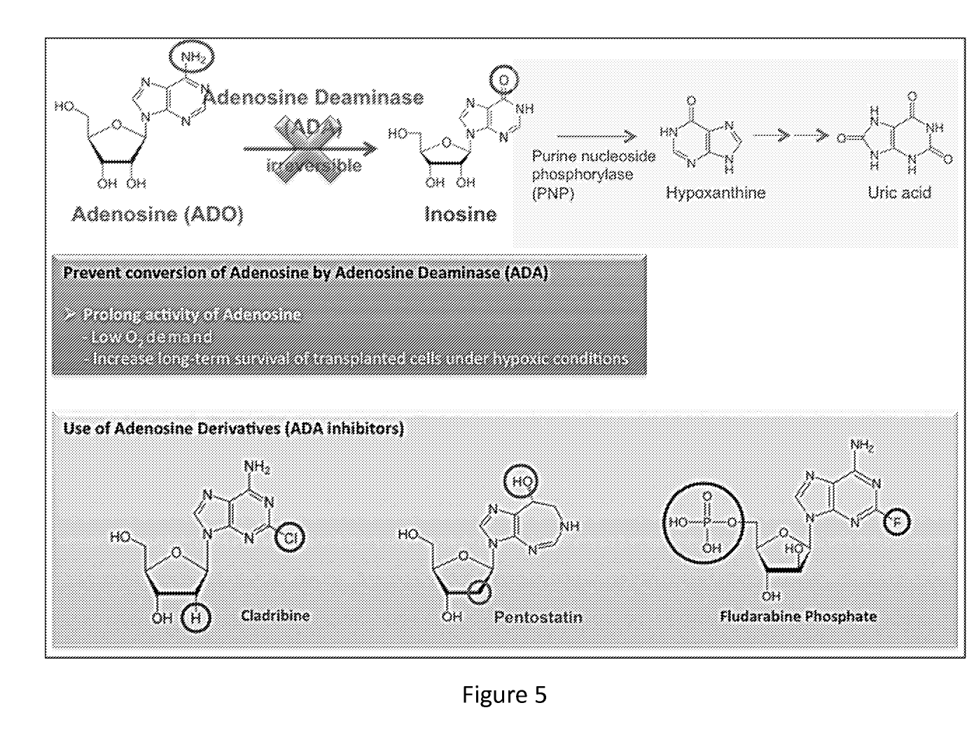

The effect of dose was also evaluated on a degree of cellular activity (FIG. 3). As the dose of adenosine increased from 1 to 10 mM, an escalation of steady hypometabolic state was maintained under hypoxic conditions, and as shown in FIG. 1, the cells were able to resume their normal metabolic activity after 7 days. The cells treated with 1 mM adenosine, however, showed a similar pattern with the cells grown in the hypoxic condition without supply of adenosine (FIG. 2). Based on this outcome, the minimum effective concentration of adenosine appears to be about 1 mM under this experimental condition. The reason for 1 mM treated cells not declining up to the duration tested may potentially have been the lower starting number of cells. This experiment demonstrated that the effects of adenosine on cellular metabolic activity are dose dependent. Finally, the long term effect of adenosine was tested on resuming its proliferation after its supply is stopped. The duration tested under the effect of adenosine was 22 days, as the angiogenesis process is known to take 2-3 weeks (Cotton, 1996, Trends Biotechnol, 14(5):158-62; Padera et al., 1996, Biomaterials, 17(3):277-84) for completion. As shown in FIG. 4, even after 22 days under the effect of adenosine, the cells applied with various adenosine doses still demonstrated the ability to restore its normal proliferation rate.

As demonstrated herein, cell viability can be maintained by down-regulating cellular metabolism under hypoxic conditions. Application of adenosine to cells under hypoxic conditions prolonged survival by decreasing the metabolic activity to a steady hypometabolic state, thus reducing oxygen demand. This concept represents a novel method for increasing cellular survival in tissue-engineered constructs during vasculogenesis.

### Example 2

### The Effect of Adenosine Derivatives on In Vitro Cell Survival

Three known ADA inhibitors were examined to evaluate the efficacies of adenosine derivatives: cladribine, pentostatin, and fludarabine phosphate. They have similar structures and molecular weights compared to adenosine, but different functional groups in the structures. Metabolic rate of cells were indirectly analyzed by cell viability in the presence or absence of adenosine derivatives and ADA in various doses. Cell viability with the drugs was evaluated under normoxic conditions.

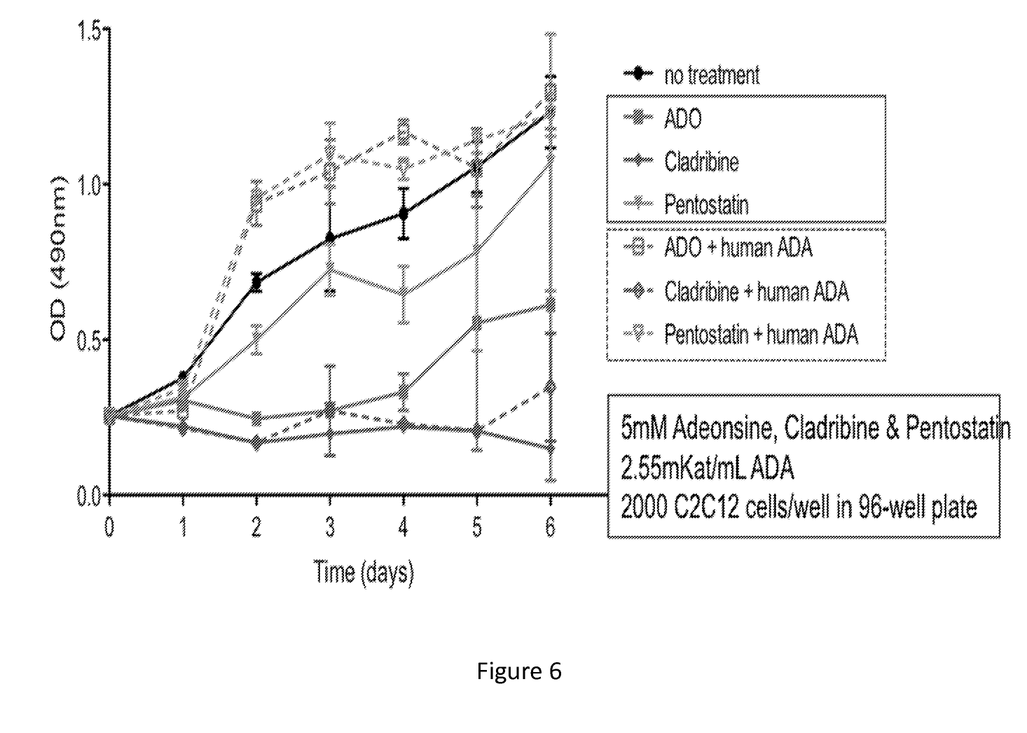

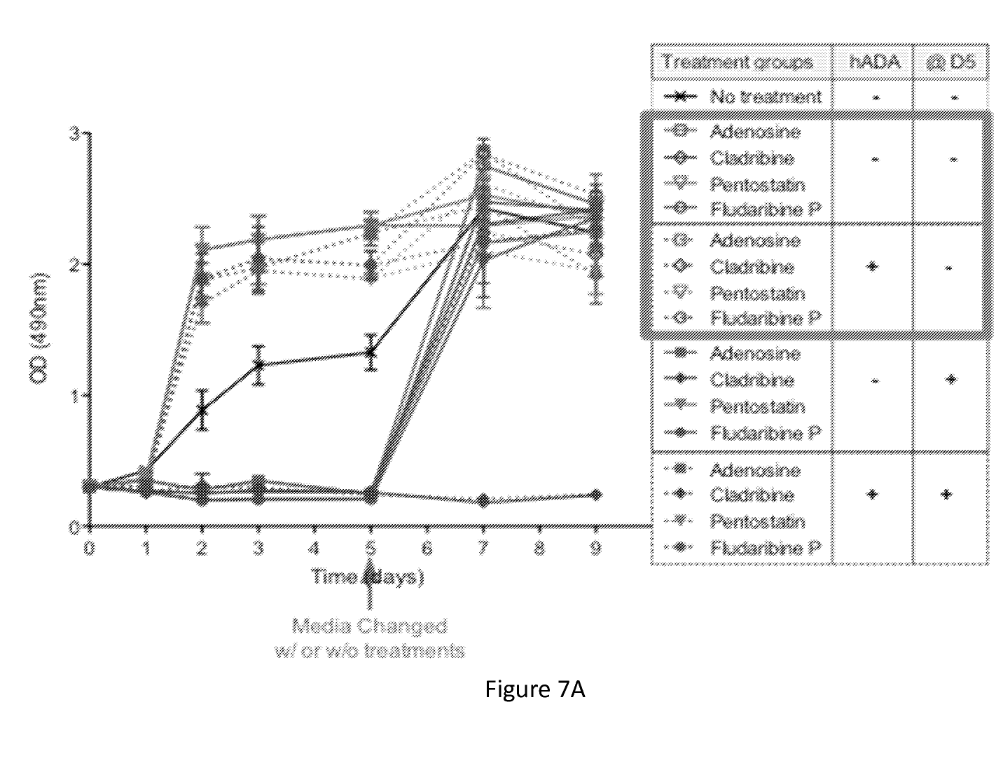

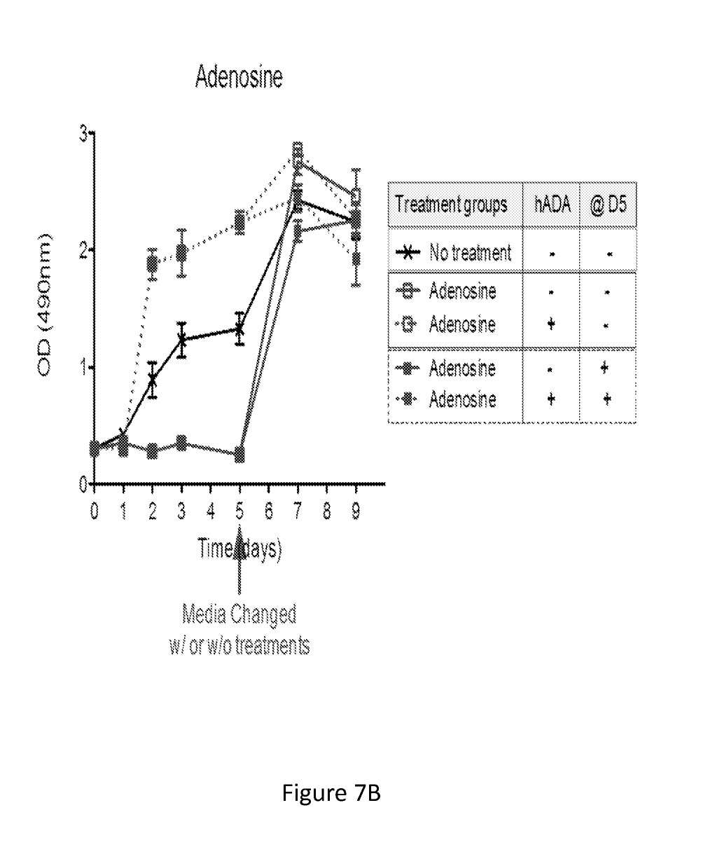

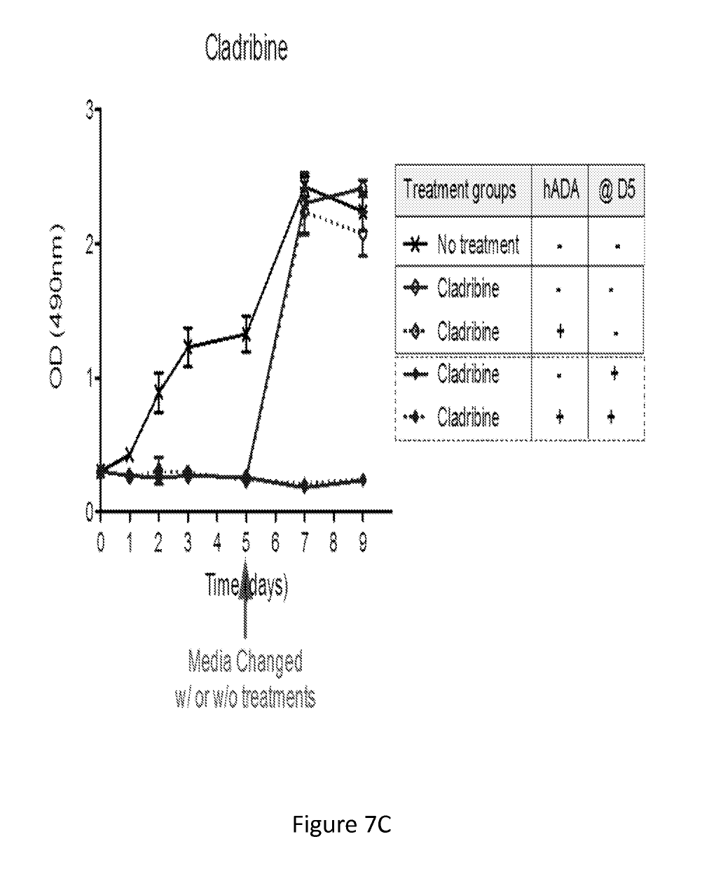

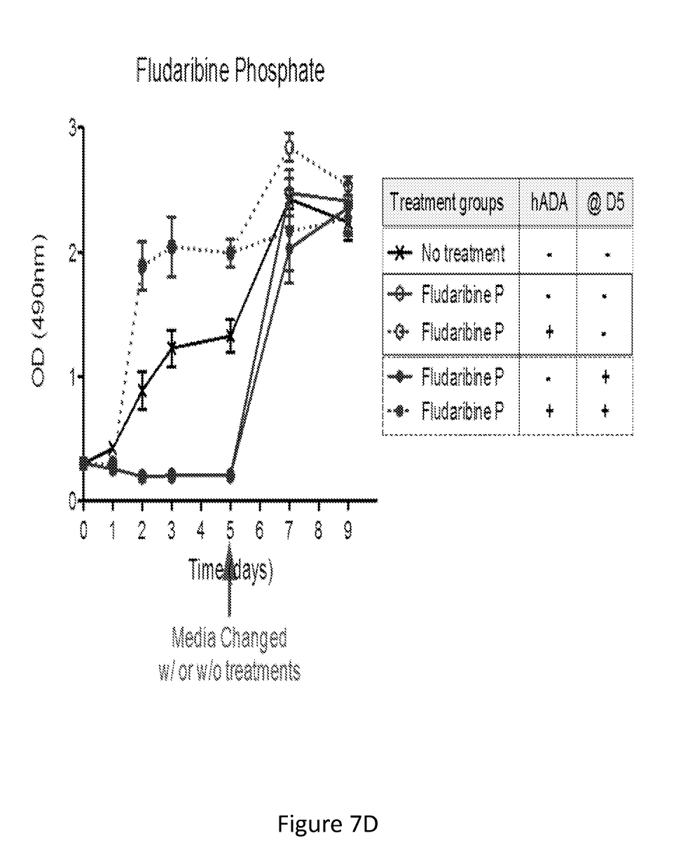

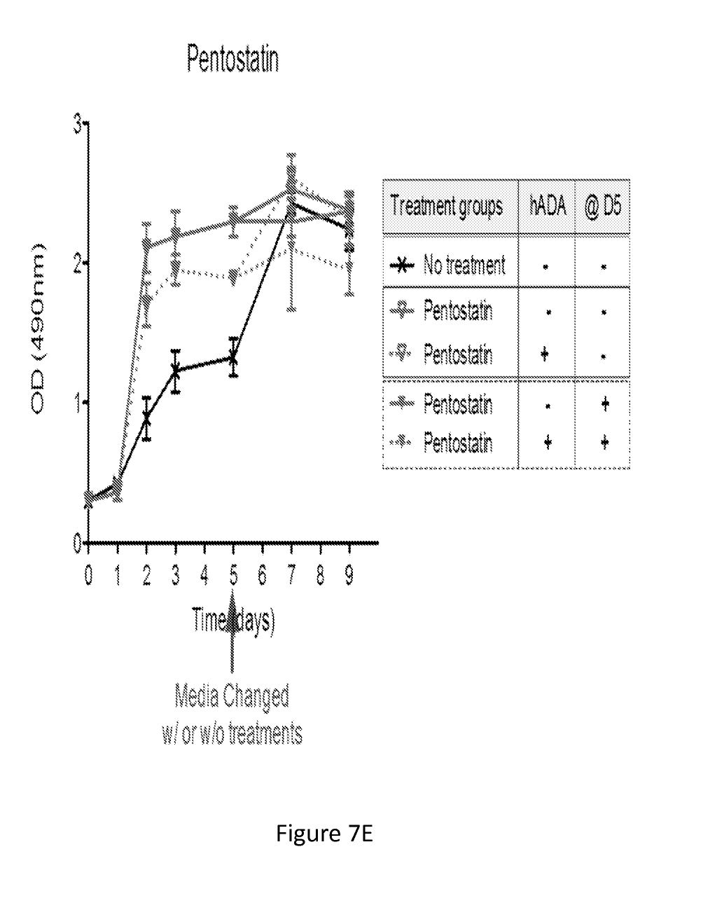

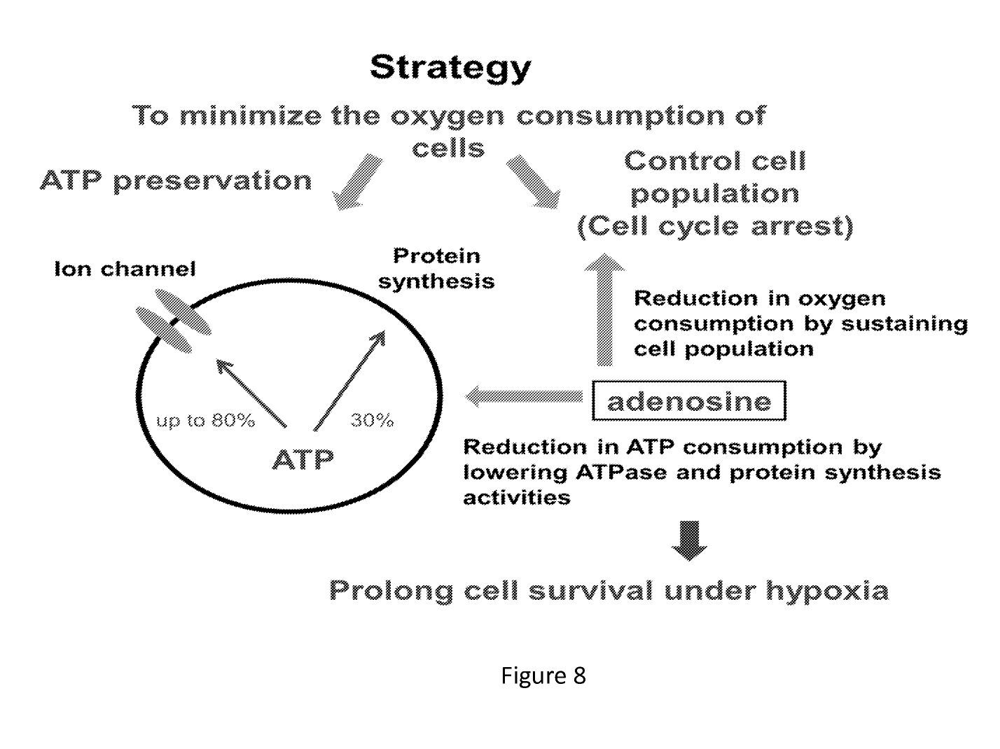

Adenosine was converted and lost its activity in presence of ADA. Among the adenosine derivatives tested, cladribine was best able to inhibit ADA and depress the cells' metabolic rate (FIG. 6). When the drugs were removed on day 5, cells recovered their relatively normal metabolic state and increased proliferation compared to non-treated cells in all groups (FIG. 7A-7E).

Among the adenosine derivatives tested, cladribine showed the most promising potential as an ADA inhibitor. It bypassed the conversion by ADA and maintained the cells in hypometabolic steady state the longest, regardless of the ADA existence. Moreover, the cladribine-treated cells recovered their normal metabolic state once the drugs were removed indicating the functional integrity of the cells.

### Example 3

### Protection Against Ischemic Injury by Metabolic Downregulation

The effect of cladribine (CDA) on tissue survival were evaluated using the following two ischemic animal models. CDA, an adenosine derivative, was used for in vivo studies as it is more stable than adenosine in the presence of adenosine degrading enzymes.

Skin flap model: The u-shaped skin flaps were created on the back of the nude mice, then silicone sheet with 100 mM CDA-containing 2% agarose gel on top of it was placed subcutaneously between muscle and skin layer (FIGS. 9A-9B). Control group received only the materials without CDA incorporated (n=3 per each group). At day 3, the flap necrosis was photographed, and H&E sections were prepared and microscopically examined.

Compartment syndrome model: Neonatal blood pressure cuffs were placed on the hind limbs of Sprague Dawley rats. A pressure of 130-140 mmHg was held for 3 hrs to induce compartment syndrome in the tibialis anterior (TA) muscle. The experimental group received 50 mM CDA in 300 uL saline daily up to day 2 after injury whereas the control group only received an equal volume of saline (n=6 per each group). The measurement of muscle tetanic force was used to assess in vivo muscle function at before injury and day 3 & 7.

All of the animals in the skin flap model study survived. No complications such as haematoma, infection or disruption of suture line developed. Photographic results revealed that the distal necrosis in flaps treated with CDA was clearly reduced compared with that in the control group in terms of skin discoloration at day 3 post-operation (FIG. 9C).

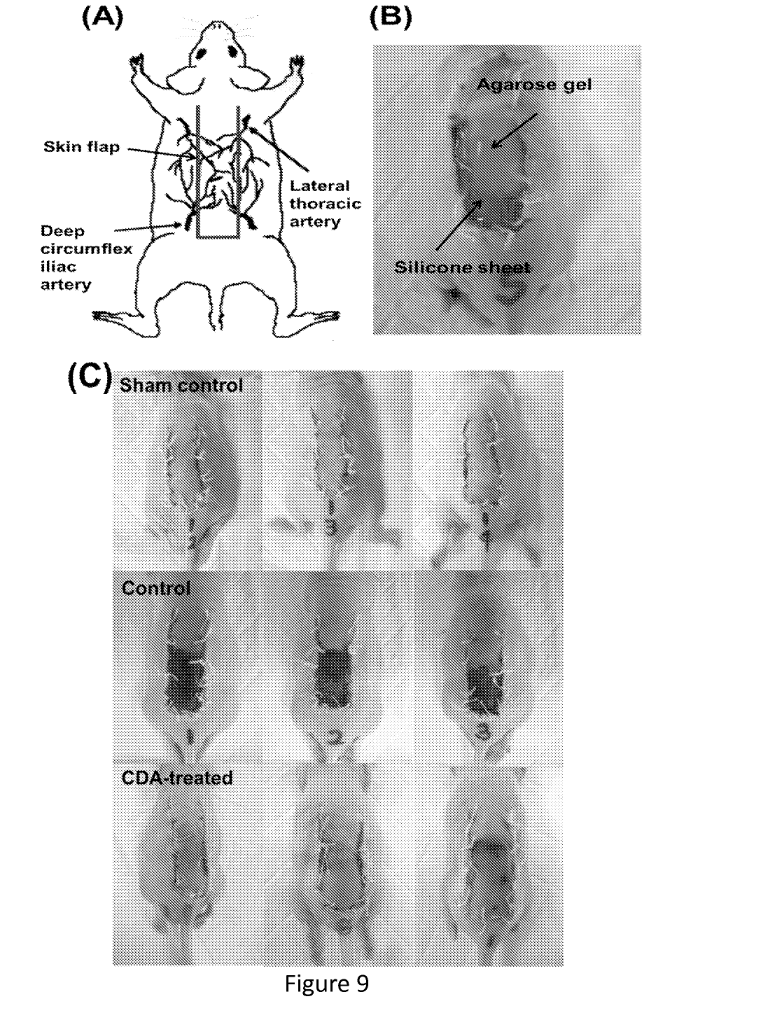

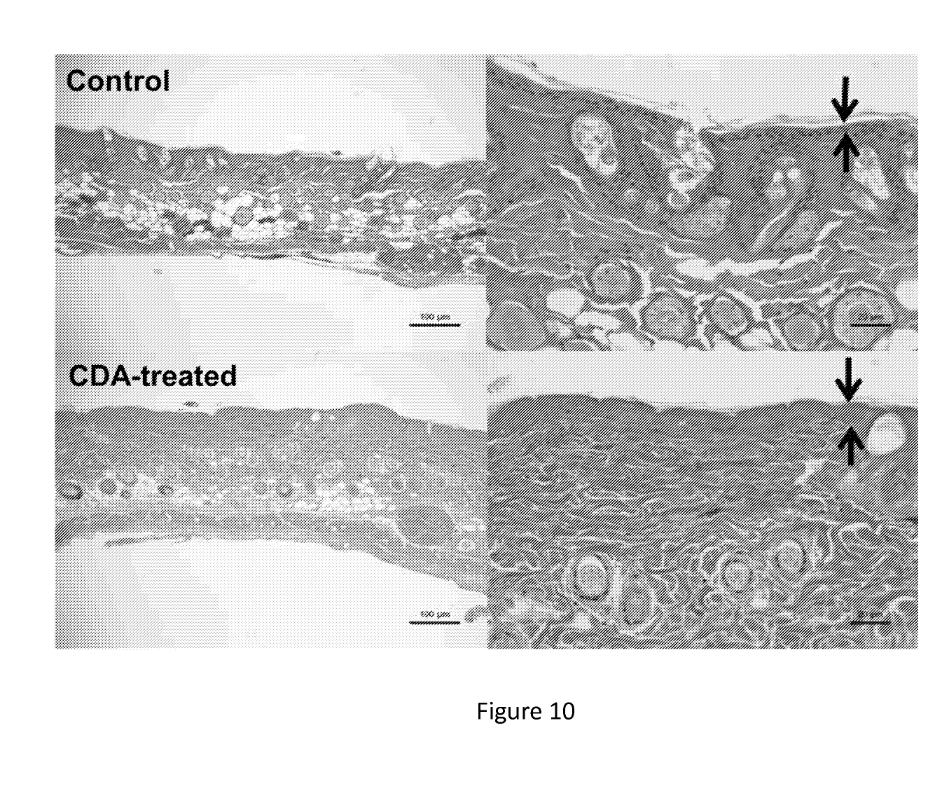

Characterization involved histological examination of tissue sections at 3 days (FIG. 10). There was a clear survival benefit for the CDA-treated group with better preservation of general tissue architecture, thickness of the skin and epidermis height. The control group had already lost much of the height in the stratified layer and dermis. In the control group, disruption of tissue architecture and indistinguishable transitions between the layers and an eosin positive mass replacing the dermis was observed. In contrast, the CDA-treated group showed a slower progression with remaining defined layers and intact epidermis.

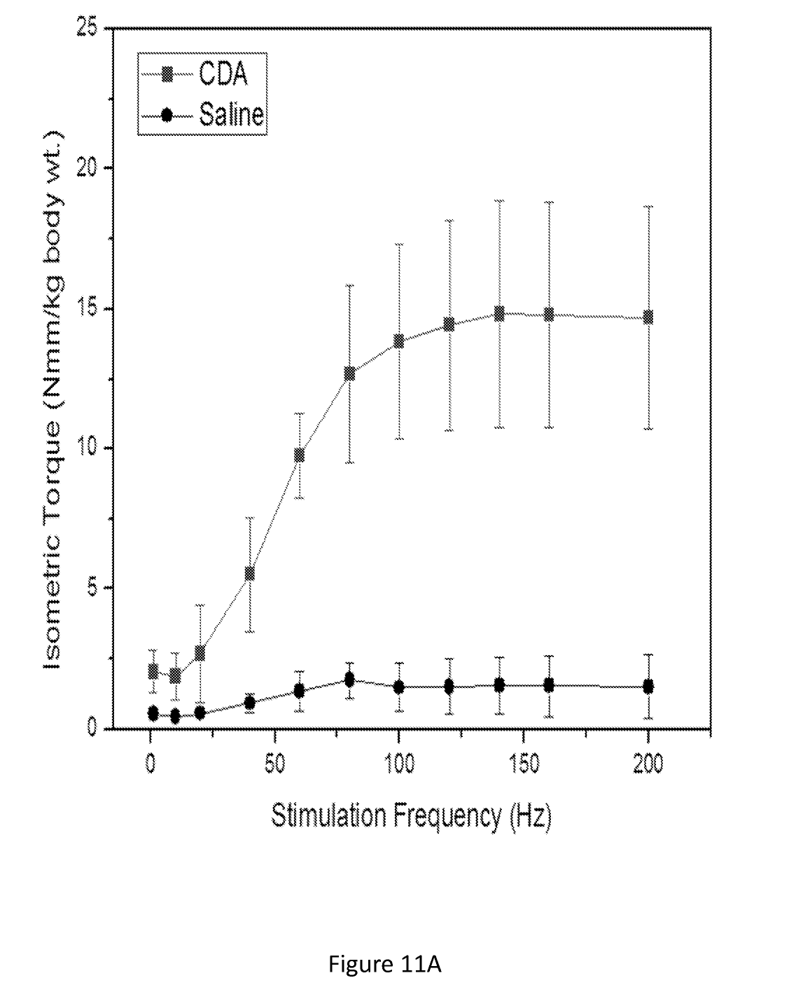

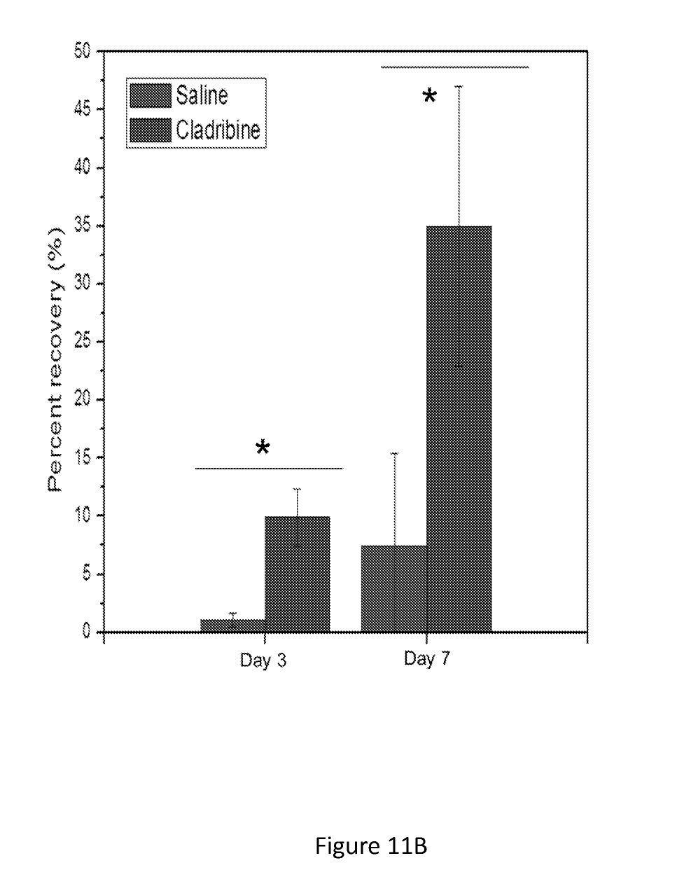

For the animals in the compartment syndrome model study, three hours of compression at 130-140 mmHg resulted in a decrease in the functional capacity of the affected TA muscles 7 days after injury (FIG. 11A). However, the anterior crural muscle twitch isometric torque, as determined via neural stimulation, significantly increased in the CDA-treated group. FIG. 11B shows the percent recovery based on the tetanic outcome at 100 Hz of the intact animals. When CDA treated, a significant recovery in muscle function was observed compared with that of control group (FIG. 11B). Approximately, 10% and 35% of the muscle function was recovered at day 3 and 7, respectively, while that of control group remained under 10% throughout.

The proposed concept represents a novel method for increasing tissue survival in necrosis or dysfunction of tissue due to insufficient supply of oxygen. In both ischemic models used in this study, the use of CDA decreased tissue necrosis (skin flap) and increased the muscle function (compartment syndrome). This indicates that improved tissue viability could be maintained at a minimum of several days by metabolic downregulation using CDA. The proposed concept represents a novel method for increasing tissue survival in necrosis or dysfunction of tissue due to insufficient supply of oxygen.

### Example 4

### Downregulation of Metabolic Activity Increases Cell Survival Under Hypoxic Conditions Applications for Tissue Engineering

Described herein is a method of sustaining cell viability under 0.1% hypoxic stress by supplying adenosine to murine myoblast C2C12 cells which lack the self-survival mechanism observed in hypoxia-tolerant cells. The cells, cultured in the presence of 5 mM adenosine, maintained their viability under hypoxia, and regained their normal growth and function of forming myotubes when transferred to normoxic conditions at day 11 without further supply of adenosine, whereas non-treated cells did not survive. An increase in adenosine concentration shortened the onset of re-proliferation after transfer to normoxic conditions. This increase correlated with an increase in metabolic downregulation during the early phase of hypoxia. A higher intracellular ATP level was observed in adenosine-treated cells throughout the duration of hypoxia. The strategy of increasing cell survival under hypoxic conditions via downregulating cellular metabolism may be useful for cell-based tissue engineering applications as well as protecting against hypoxic injuries

The materials and methods employed in these experiments are now described.

### Cell Culture

C2C12 murine myoblasts (ATCC) were cultured in a growth medium containing Dulbecco's modified Eagle's medium (DMEM, Gibco) supplemented with 10% FBS, 500 U/mL penicillin and 500 μg/mL streptomycin.

### Hypoxic Treatment

At 60-80% confluency under normal conditions, 100 μL of a cell suspension containing 2,500 cells was plated into each well of a 96-well plate. Cells were incubated for 24 h in normoxic conditions (21% O₂, 37° C.) prior to placement in a hypoxic chamber to allow time for attachment to the culture plates. Hypoxic condition was maintained with a gas mixture containing 0.1% O₂, 5% CO₂ and 94.9% N₂ at 37° C. and full humidity in an X-Vivo System (Biospherix). At day 0, each group was supplemented with a fresh medium where adenosine groups received various concentrations of adenosine (0.05, 1, 2 and 5 mM, Sigma-Aldrich) dissolved in the medium. The hypoxic groups were then placed to the hypoxic chamber and incubated up to day 11, whereas the normoxic group continued to be incubated in the regular incubator. No medium change was made on both normoxic and hypoxic groups up to day 11. After transfer to normoxic conditions, cells were cultured in the regular incubator with an exchange of no adenosine-containing, fresh medium every third day.

### Cell Counts

Cell proliferation of each group was determined by a Quanti-iT PicoGreen dsDNA Kit (Invitrogen). After washed with PBS, cells were lysed with 55 μL of RIPA buffer (Sigma) on ice for 5 min, then the supernatants were mixed with an equal volume of PicoGreen dsDNA quantitation reagent, a fluorescent DNA dye (excitation at 480 nm; emission at 520 nm). The DNA content was quantified using a SpectraMax M5 microplate reader (Molecular Devices). The number of cells was calculated using a standard curve plotted from fluorescent readings of serial dilutions of a known concentration of cells.

### Viable Cell Imaging

Cell viability was visualized using a calcein AM (Invitrogen) where viable cells fluoresce green through the reaction of calcein AM. Cells were rinsed with PBS and incubated for 30 min in a PBS composed of 2 μM calcein AM. Then, the cells were observed under an inverted fluorescent microscope (Leica).

### Histological Analysis for Myotube Formation

After C2C12 cells that survived the hypoxic stress in the presence of adenosine were transferred to normoxic conditions without further supply of adenosine, they were cultured continuously for 3 days in the growth medium followed by 2% horse serum-containing medium for another 6 days. They were then fixed with methanol and immunostained with a monoclonal antibody directed against the muscle sarcomeric myosin (MF-20, 1:25, abcam) followed by an exposure to secondary antibody (anti-mouse alexa 594, 1:500, abeam). Nuclei were counterstained with DAPI (Vector). Photomicrographs were obtained using a Leica inverted fluorescence microscope. Myotube formation was also observed by Giemsa staining (1:20, Sigma-Aldrich) using a Zeiss upright light microscope.

### MTS Metabolic Assay

Mitochondrial metabolic activity of viable cells was assessed using a 3-(4,5-dimethylthiazol-2-yl)-5-(3-carboxymethoxyphenyl)-2-(4-sulfophenyl)-2H-tetrazolium (MTS) assay (Promega). Cells were rinsed with PBS three times followed by the addition of 120 μL of MTS reagent to each well. After an hour of incubation, optical density (OD) of a brown formazan product by dehydrogenase enzymes in metabolically active cells was measured with a microplate reader at 490 nm. The mean OD value obtained from media blanks was standardized as 0% metabolic inhibition. For some analyses, metabolic activity measured at each time point was normalized by the cell number obtained from PicoGreen assay as previously described. The total metabolic activity represented by the area under the curve over time was computed using Origin Pro v. 8 (Origin Lab Corporation).

### Measurement of Intracellular ATP Level

Intracellular ATP was measured by using the ATP Bioluminescence Assay Kit HS II (Roche Applied Science). Cells were lysed with 50 μL of lysis buffer and transferred into a well, then 50 μl of luciferase reagent was added to it. After mixing, the light emitted was measured and integrated for 10 seconds by using a SpectraMax M5 luminometer (Molecular Devices). The blank value (from a well containing no ATP) was subtracted from each sample's raw data. ATP concentrations were calculated from the linear part of the standard curve prepared with serial dilutions of a known concentration of ATP and expressed as moles per cell. This, in turn, was re-calculated to a percent ATP of that expressed in the cells grown under normoxic condition.

### Survival of Muscle Tissue Under Hypoxia

Soleus muscle tissues dissected immediately after sacrifice of Sprague Dawley rats (17 weeks old, 380-400 g, Harlan) were assigned to one of three groups in the growth medium: (1) native tissue group (2) no adenosine-treated hypoxic group and (3) hypoxic group with 5 mM adenosine supplemented in 2 mL of medium. The media was changed every third day. At day 10, a half of the tissue sample was fixed in 10% neutral buffered formalin (Sigma-Aldrich), 6 μm sections were generated by a cryotome (Leica), and stained with hematoxyline and eosin (H&E). Microscopic analysis was performed using a light microscope (Zeiss). Dead assay was assessed on the remaining tissues using 4 μM ethidium homodimer-1 (EthD-1, Invitrogen) to stain dead cells with damaged cell membranes. The stained sections were observed using a fluorescent microscope (Leica). The number of dead cells was quantitated using the “analyze particle” method with Image J software (U.S. National Institute of Health) on the fluorescence images, and its percentage was calculated based on the number of DAPI-stained cells on each section.

### Statistical Analysis

Statistical analysis was performed using a single-tailed Student's t-test and one-way ANOVA with Tukey's post hoc tests (Origin Pro v.8, Origin Lab Corporation). A P value <0.05 was considered significant. All values were reported as the mean and standard deviation of the mean.

The results of the experiment are now described.

### Adenosine Enhances Cell Survival Under Hypoxia

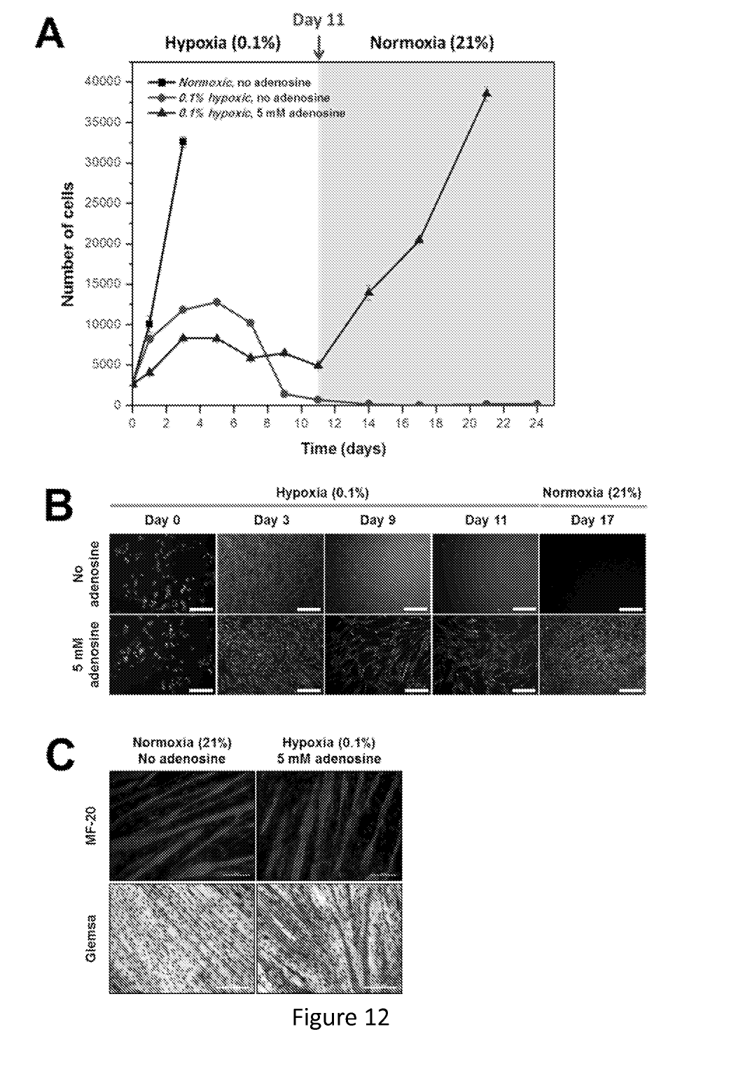

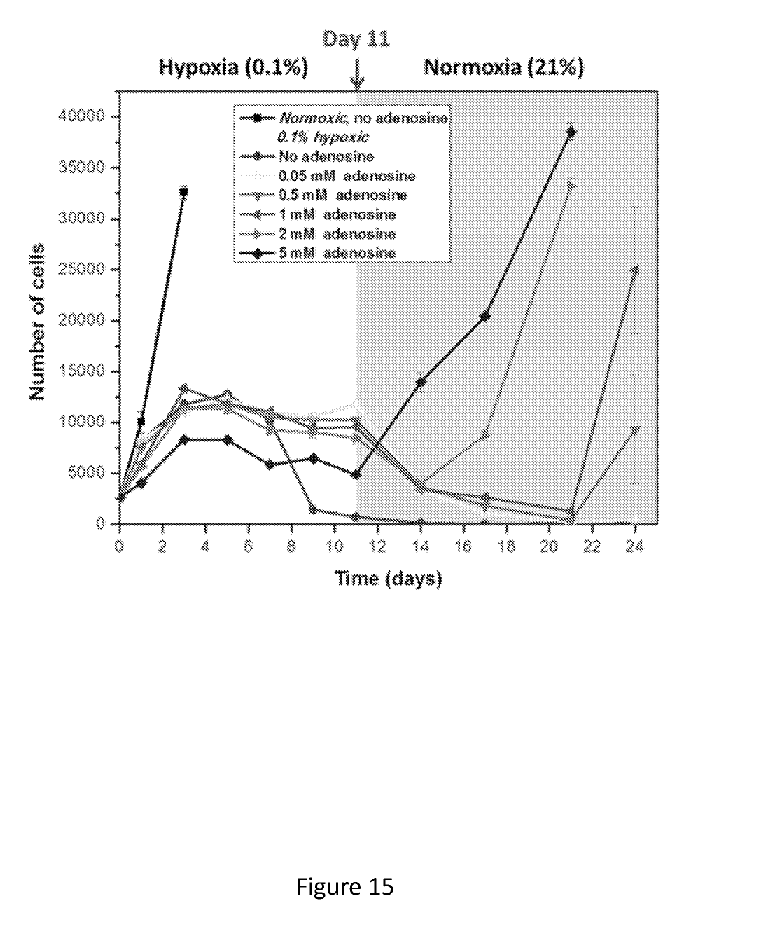

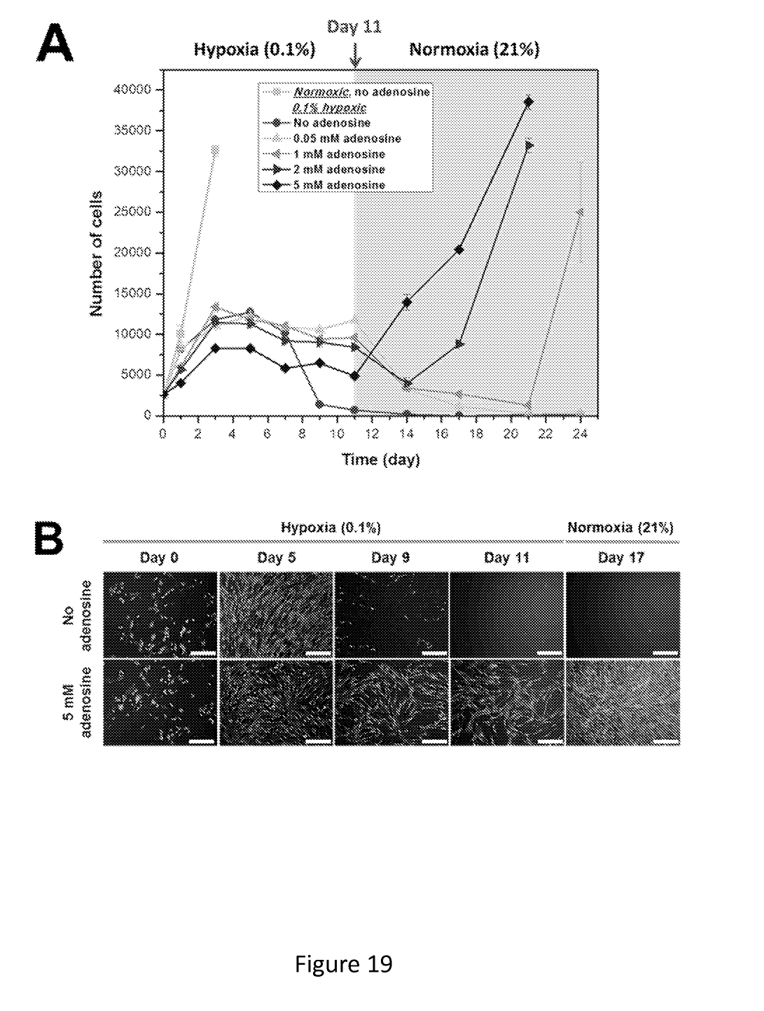

The effect of adenosine on C2C12 cell survival under hypoxia was investigated by culturing cells under 0.1% hypoxic conditions for 11 days followed by normoxic conditions without further supply of adenosine. The normoxic cells became fully confluent at day 3 (FIGS. 12A, 15, 19), and then their number declined. The growth of all hypoxic groups was substantially limited under 0.1% oxygen conditions. Hypoxic cells not treated with adenosine showed an increase in number up to day 5, but then continued to decline and became almost necrotic at day 11 with only 5.5% of the initial number of cells remaining viable. Growth of these cells was never recovered even after transfer to normoxic conditions. This was also observed in 0.05 mM adenosine-treated cells, however, cells exposed to 1, 2 and 5 mM adenosine survived 11 days of hypoxic stress, and still maintained approximately two to four times of the initial number of cells. These observations were also supported by the fluorescent images of live C2C12 cells stained with calcein AM (FIGS. 12B, 19B). Also, these cells resumed their proliferation at a growth rate comparable to that in normoxic cells after transfer to normal oxygen tension. The time for onset of re-proliferation was found to be concentration-dependent: the higher the adenosine concentration the shorter the time to initiation of cell growth. An effect of concentration on cell number was also revealed under hypoxia, however, no substantial differences in number of cells were observed among the groups except for the 5 mM adenosine-treated group. Since the most effective adenosine concentration was 5 mM, this concentration was used for further experiments.

C2C12 Cells Surviving in the Presence of Adenosine Retain their Differentiating Property

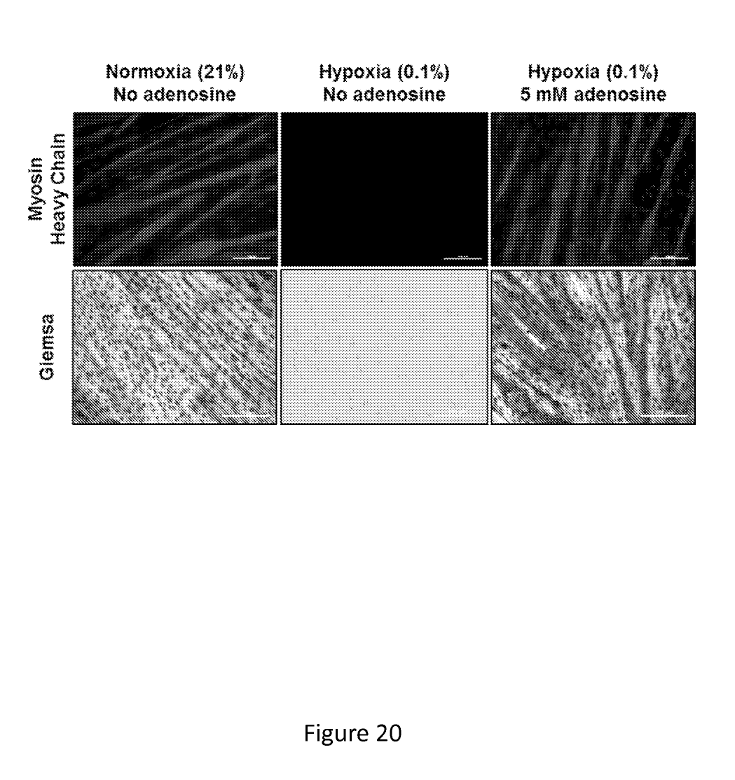

It is critical that cells retain their normal function after the effect of adenosine is removed. Exposure to adenosine did not affect the proliferative capability of C2C12 cells (FIGS. 12A, 15, 19). Another important function of cells, especially for tissue engineering applications, is their differentiating capability. C2C12 cells possess a unique property of differentiating into myotubes in the 2% horse serum-containing medium. Using this property, the differentiating capability was qualitatively evaluated on C2C12 cells that underwent 11 days of exposure to adenosine under hypoxic conditions. C2C12 cells cultured under normoxic conditions were used as a control. Adenosine-treated C2C12 cells showed capability of forming myotubes by fusing and becoming lined up with their elongated cytoplasmic extensions (FIGS. 12C, 20). Moreover, they revealed a typical culture of multicellular myotubes compared with those obtained from the control cells in terms of the number, length and thickness. In the cells not treated with adenosine, only the cellular debris was observed without myotubes formed.

### Mechanism Behind Cell Survival: Adenosine Maintains Hypometabolic Steady State of Hypoxic C2C12 Cells

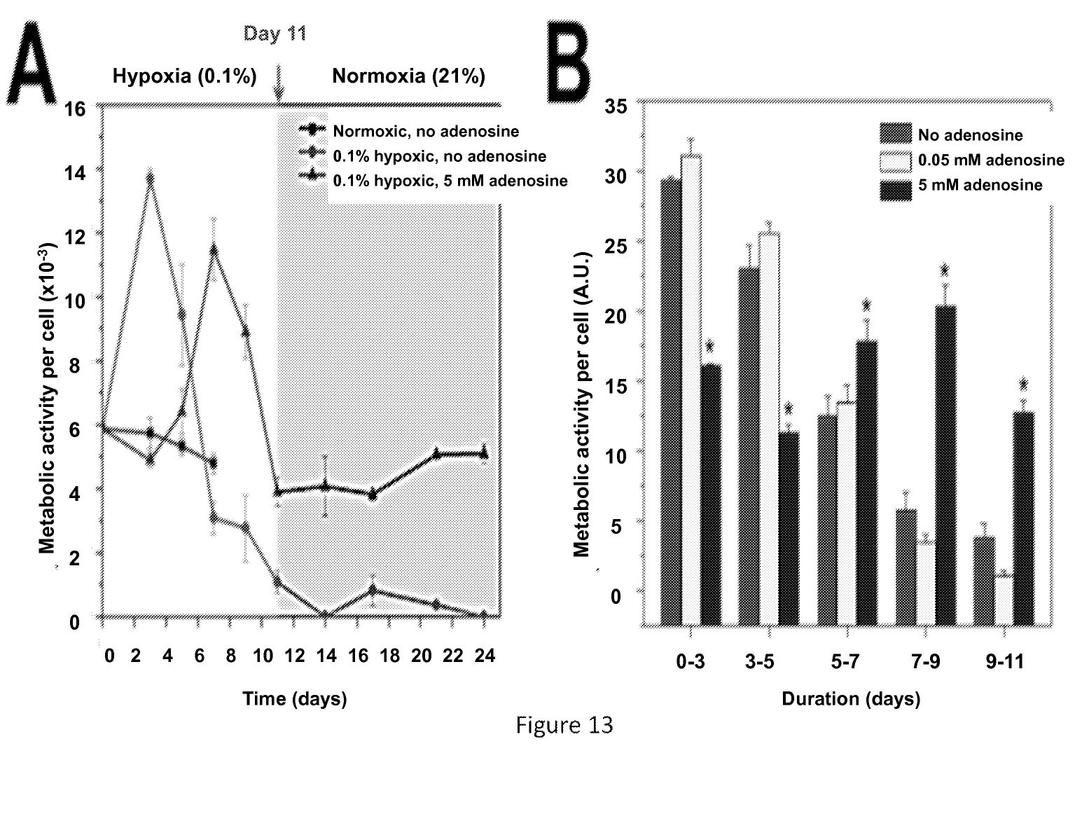

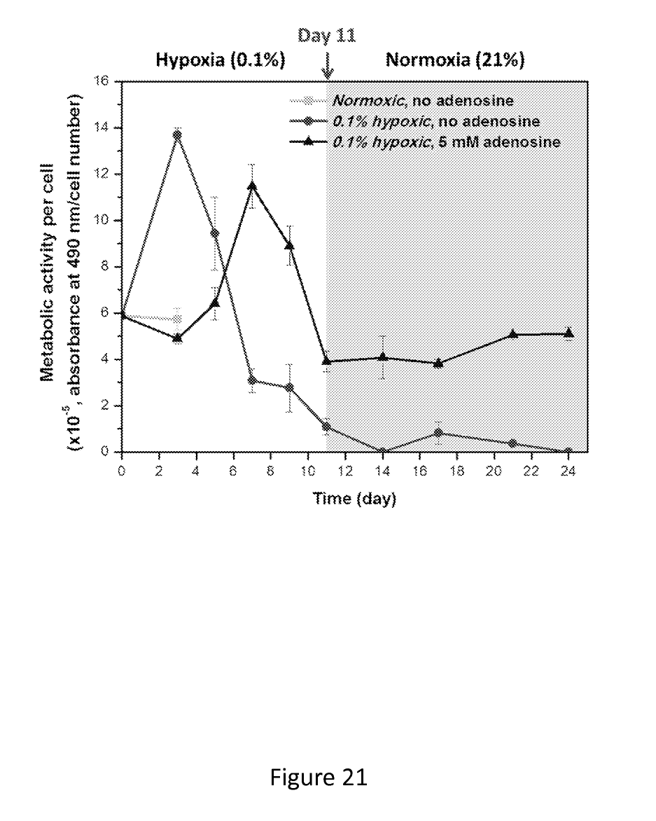

It was examined whether the downregulation of metabolic activity was observed in the surviving C2C12 cells under hypoxic conditions by the presence of adenosine. Metabolic activity was presented as MTS absorbance normalized by the number of cells at each corresponding time point. The metabolic activity of the hypoxic cells without adenosine increased initially, but then, decreased and never recovered even after transfer to normoxic conditions (FIGS. 13, 21). In the cells treated with 5 mM adenosine, however, metabolic activity was suppressed initially up to day 5, and then showed a transient increase followed by a decrease, while still maintaining approximately 68% of the initial metabolic activity at day 11. After transfer to normoxic conditions, metabolic activity was restored to a level equivalent to that in normoxic cells.

### A Higher Intracellular ATP Level is Observed in the Adenosine-Treated Cells

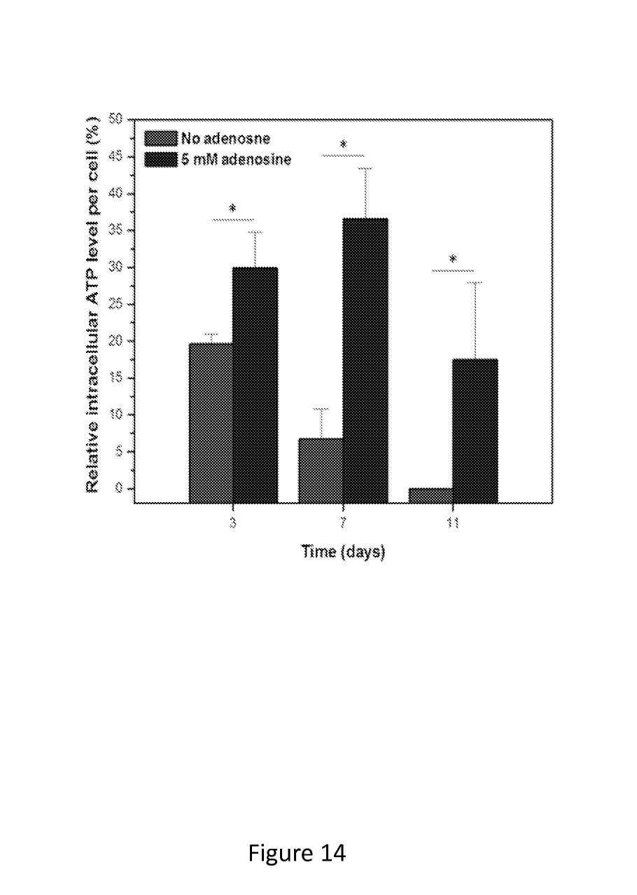

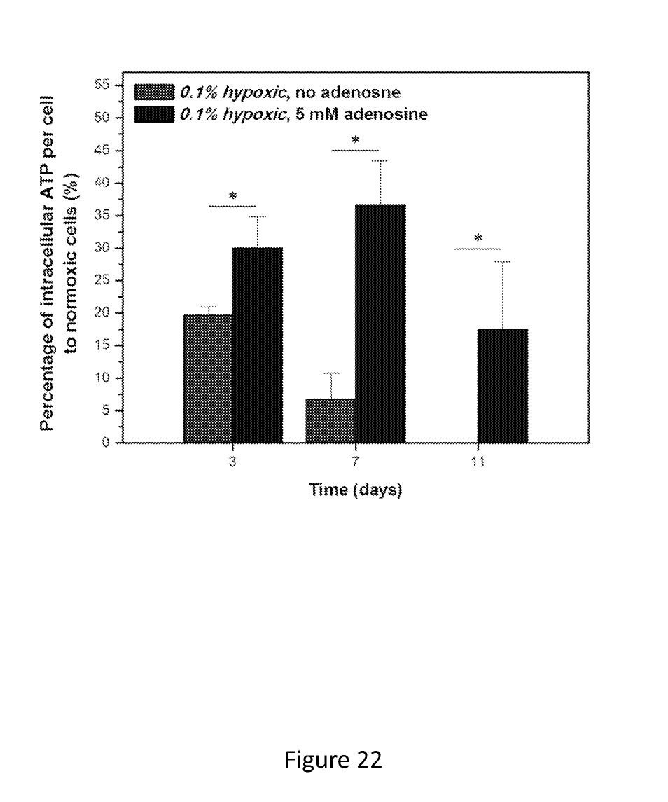

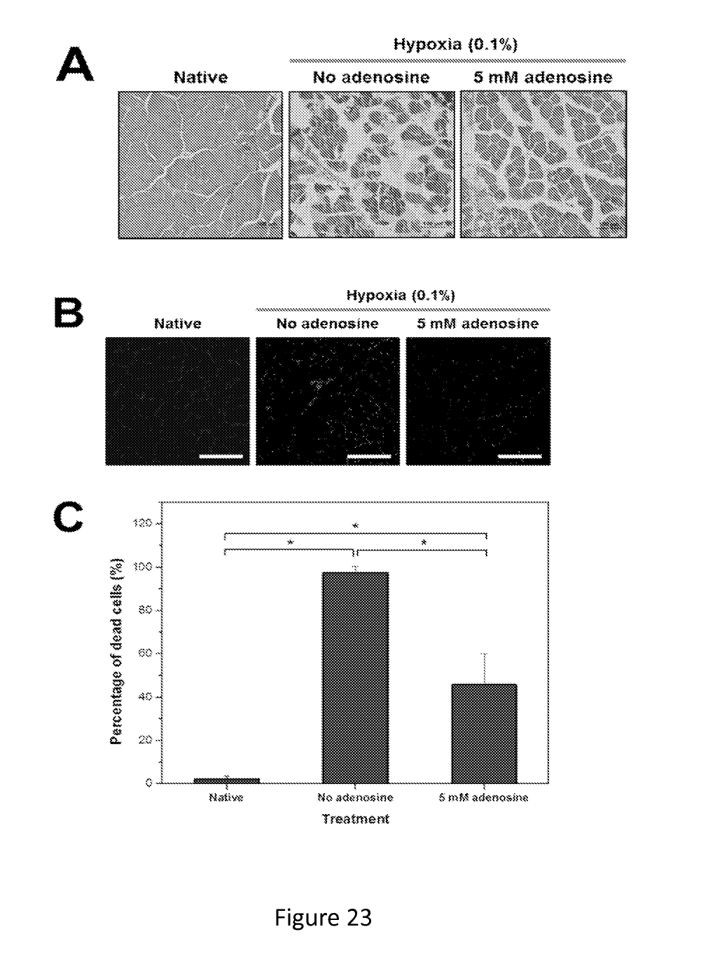

The effect of adenosine on intracellular ATP level during the hypoxic phase was examined. As described elsewhere herein, the ATP level during the hypoxic phase was also normalized by the number of cells, and this, in turn, was expressed as a percentage of the ATP level measured in normoxic cells (FIGS. 14, 22). In the no adenosine-treated control cells, an 81% reduction in ATP level was observed by day 3. This decrease continued to 93% by day 7, and ATP was no longer detected by day 11. In contrast, a consistently higher ATP level (p<0.05) was observed throughout the hypoxic duration in the cells treated with adenosine than the control cells, with 18% ATP still detected at day 11.

### Adenosine Provides Tissue Protection Under Hypoxia

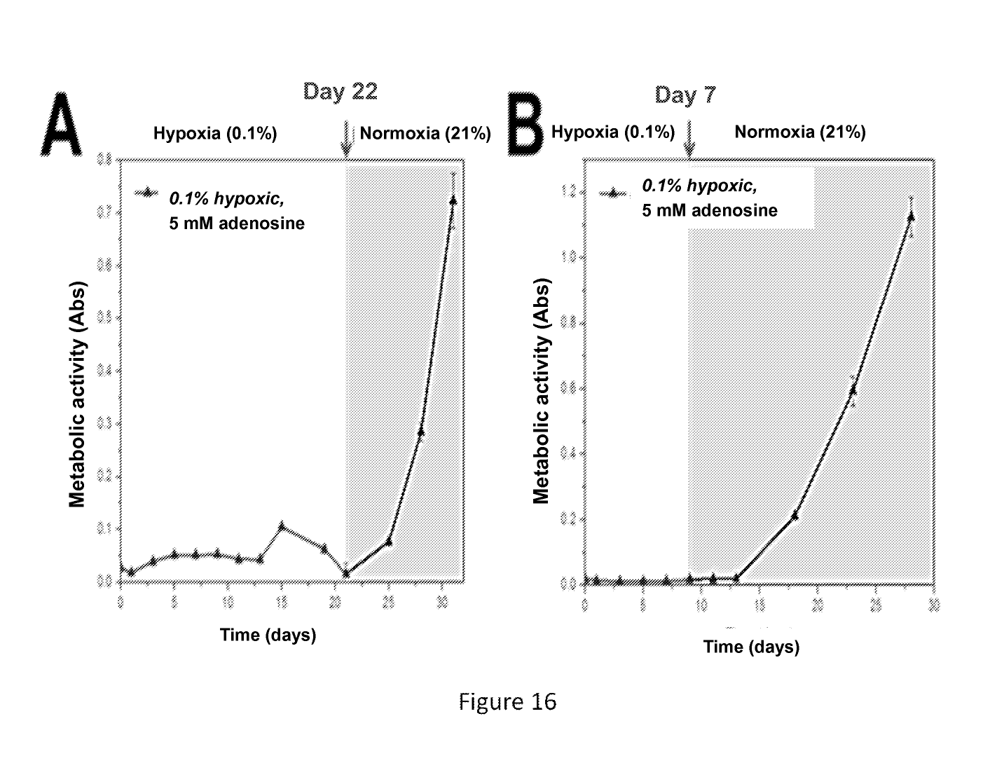

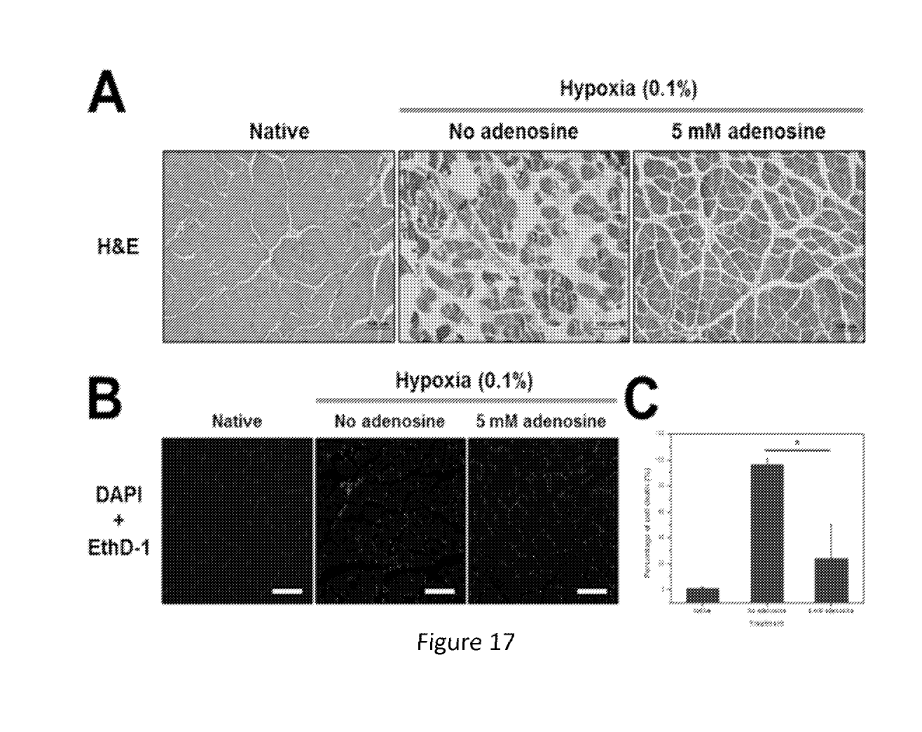

The effect of adenosine on prolonging cell survival may be useful for protecting against hypoxic injury (FIG. 16). The effect of prolonging cell survival using adenosine was examined using soleus muscles, which are primarily aerobic, by culturing these muscles under hypoxic conditions for 10 days. The degree of muscle damage with H&E was assessed (FIGS. 17A, 22A). Native muscle tissues had no histologic evidence of injury. The tissues cultured without adenosine showed more damage than those treated with 5 mM adenosine, indicated by degenerated myofibers and loss of connective tissues. It was evaluated whether a degree of damage correlates with the number of dead cells. Fluorescent images of cross-sectioned muscle tissues immunostained with ethidium homodimer-1 (EthD-1) and DAPI showed that a significant number of cells died in the no adenosine-treated tissues, whereas, in the presence of adenosine, a pronounced reduction in cell death was observed (FIGS. 17B, 22B). Few or no dead cells were observed in the native tissues. This observation was more evident when the number of dead cells was quantified and expressed as a percentage of number of DAPI-stained cells (FIGS. 17C, 22C). The no adenosine-treated group revealed 97% cell death, while only 46% dead cell was shown in the tissues supplied with adenosine (p<0.01). Although not wishing to be bound by any particular theory, this result suggests that adenosine can also be effective protecting hypoxic muscles by reducing cell death.

### Example 5

### Induction and Maintenance of Hypometabolism for Tissue Protection Against Ischemia

As described herein, ADA inhibitors have been employed as adenosine replacements to induced hypometabolism and prolonged cell survival. Using an ADA inhibitor, stable metabolic downregulation and long-term cell survival were successfully achieved in vitro. Furthermore, in vivo ischemic damage was delayed in skin flap and functional recovery of rat tiabialis anterior (TA) muscle was accelerated. Hypometabolism induced by ADA inhibitor may be useful for clinical tissue protection against ischemic injury.

The materials and methods employed in these experiments are now described.

### Adenosine and ADA Inhibitors

Adenosine (Sigma-Aldrich), cladribine (Sigma, 2-Chloro-2′-deoxyadenosine), pentostatin (Tocris biosciences), and fludarabine phosphate (LGM Pharma) were used throughout this study. Adenosine deaminase (ADA) was treated in combination with adenosine and adenosine derivatives to mimic the function of physiological ADA. Human adenosine deaminase (ADA1, Sigma) was used for cell metabolic measurements in normoxia and recombinant human adenosine deaminase (rhADA, R&D systems) were used for the rest of the study due to the supply shortage of human ADA1. The amount of ADA was calculated to inactivate 5 mM of adenosine over a desired number of days.

### Cell Culture

C2C12 mouse myoblasts (ATCC) were cultured in Dulbecco's Modified Eagle's Medium with high glucose (Gibco) supplemented with 10% fetal bovine serum (Hyclone) and 1% penicillin-streptomycin (100 units penicillin; 100 μg streptomycin/ml, Gibco). The cells were maintained at 37° C. in a humidified, 5% carbon dioxide atmosphere for normoxic condition. For hypoxic incubation, the chamber (Xvivo System, Biospherix) was maintained at 37° C. in a humidified 5% carbon dioxide and 1% oxygen atmosphere.

### Hypoxic Treatment

100 μL of a cell suspension containing 2,500 cells was plated into each well of a 96-well plate. Cells were incubated for 24 hours in normoxic conditions (21% O₂, 37° C.) prior to placement in a hypoxic chamber to allow attachment to the culture plates. Hypoxia was induced by incubating cells at 37° C. and full humidity in an X-Vivo System (Biospherix) maintained with a gas mixture containing 0.1% O₂, 5% CO₂ and 94.9% N₂. At day 0, each group was supplemented with a fresh medium with 5 mM of adenosine, cladribine, or fludarabine phosphate, and ADA dissolved in the medium. During the hypoxic phase, medium was not changed except for 7 days. The cells were then transferred to the normoxic chamber, with change of new media with or without ADA inhibitors and ADA.

### Metabolic Rate Measurement

Cellular metabolic activity was evaluated using MTS (3-(4,5-dimethylthiazol-2-yl)-5-(3-carboxymethoxyphenyl)-2-(4-sulfophenyl)-2H-tetrazolium, inner salt, Promega) assay. This colorimetric method relies on NAD(P)H-dependent oxidoreductase enzymatic activity to reduce MTS into soluble formazan in presence of PMS (phenazine methosulfate). Briefly, MTS stock solution (2 mg/mL in PBS) and PMS (0.92 mg/mL in PBS) were mixed with 20:1 ratio and added into culture wells with 1:5 ratio (reagent:media). The plate was incubated for one hour at 37° C. and the absorbance measured at 490 nm (SpectraMax M5, Molecular Devices).

### Intracellular ATP Content Measurement

Cellular ATP content was measured using ATP Bioluminescence Assay Kit HSII (Roche) following the manufacturer's instructions. It utilizes the light emitting luciferase-catalyzed oxidation of luciferin to measure low concentration of ATP. Briefly, the cells were lysed using lysis reagent and centrifuged at 1500 rpm for 5 minutes. Supernatant was collected and measured for intracellular ATP content with luciferase reagent at maximum emission of 562 nm (SpectraMax M5, Molecular Devices).

### Live and Dead Cell Analysis

Cells were analyzed for live and dead population using Live/dead viability/cytotoxicity kit (Molecular Probes), which stains live cells fluorescent green with calcien AM and dead cells fluorescent red with Ethidium homodimer-1. Cells were rinsed with PBS and incubated for 30 min in a PBS composed of 1 μM calcein AM and 2 μM EthD-1 final concentration. Then, the fluorescence were measured (ex/em at 490 nm/530 nm for calcein AM and ex/em at 530 nm/645 nm for EthD-1, SpectraMax M5, Molecular Devices) and cells were observed under an inverted fluorescent microscope (Leica Axiovert).

### In Vitro Tibialis Anterior (TA) Muscle Tissue Survival

Rat tibialis anterior (TA) muscle tissues were dissected immediately after sacrifice of Sprague Dawley rats (17 weeks old, 380-400 g, Harlan), injected with blank, 5 mM of adenosine or cladribine, and then incubated in 5 mL of complete media for 3 days.

### Histological Analyses for In Vitro Tissue Incubation

The tissue samples were fixed in 10% neutral buffered formalin (Sigma-Aldrich), 6 μm sections were generated by a cryotome (Leica), and stained with hematoxyline and eosin (H&E) and with TUNEL staining. Microscopic analyses were performed using a light microscope (Zeiss M1) and ImagePro software.

### Skin Flap Model

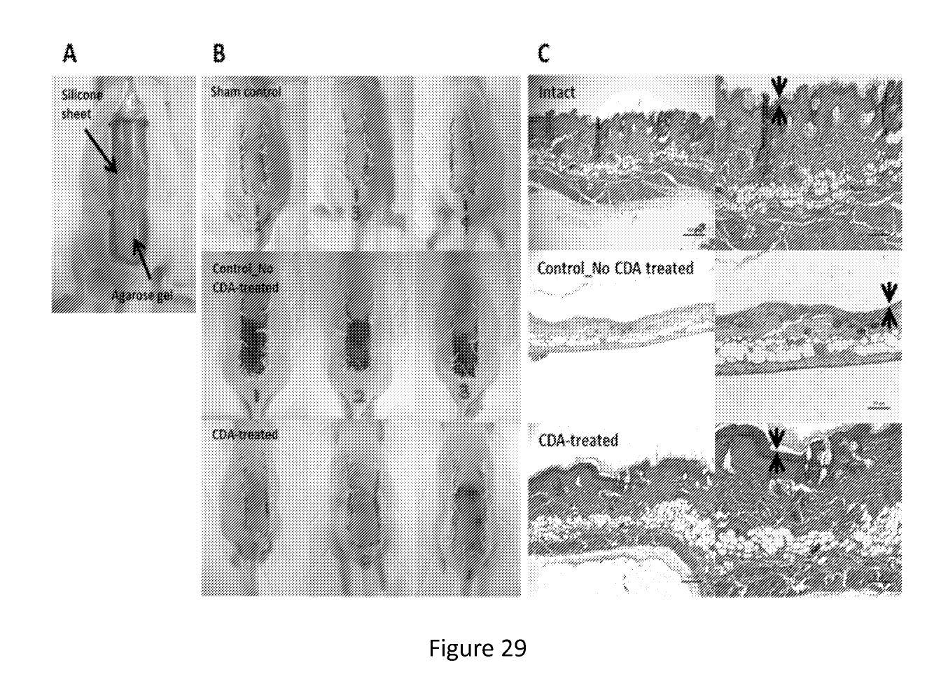

The u-shaped skin flaps were created on the back of the nude mice, then silicone sheet with 100 mM CDA-containing 2% agarose gel (A9045, Sigma) on top of it was placed subcutaneously between muscle and skin layer (FIG. 29A). Control group received only the materials without CDA incorporated (n=3). At day 3, the flap necrosis was photographed, and H&E sections were microscopically examined.

### Compartment Syndrome Injury Model

All animal studies were performed in strict compliance with Wake Forest University IACUC and NIH guidelines. Male Lewis rats (6-8 weeks old, Harlan) were anesthetized with 2-3% isofluorane prior to all procedures. A #2 sized neonatal blood pressure cuff (Trimline Medical Products Tempa-Kuff) was tightened around the hind limb proximal to the extensor digitorum longus (EDL) muscle. The time and amount of pressure used varied as indicated. Due to the extended length of anesthesia, rats were administered IP injections of saline over the course of the procedure. Rats were euthanized at 1, 2, 4, 7, 14 or 35d post injury. At least 3 separate experiments were analyzed.

Neonatal blood pressure cuffs were placed on the hind limbs of Sprague Dawley rats. A pressure of 130-140 mmHg was held for 3 hours to induce compartment syndrome in the tibialis anterior (TA) muscle. The experimental group received injection of 30 mM cladribine in 200 μL saline daily up to day 2 after injury whereas the control group only received an equal volume of saline (n=6). The measurement of muscle tetanic force was used to assess in vivo muscle function at before and right after injury, day 3, 7, 11 and 14.

### Histology and Immunofluorescence for In Vivo Ischemic Models

Muscle tissues were harvested and weighed, fixed in 10% neutral buffered formalin for 24 hours and embedded in paraffin. Serial paraffin sections (8 μm) were analyzed using hematoxylin and eosin (H&E) staining for morphology and Masson's Trichrome stain to examine collagen deposition and fibrosis. Wheat germ agglutinin (WGA) Alexa Fluor 594 (Invitrogen, Carlsbad, Calif.) was used to label cell membranes. α-bungarotoxin-594 (BTX, Invitrogen, Carlsbad, Calif.) was used to label acetylcholine receptors in neuromuscular junctions (NMJs). The desmin (sc-23879) and MyoD (sc-304) antibodies were obtained from Santa Cruz (Santa Cruz, Calif.) and the Pax 7 and myogenin (F5D) antibodies were obtained from the Developmental Mouse Hybridoma Bank. Images were acquired with a Leica DM400B upright fluorescent microscope and a Retiga-2000RV Qimaging camera.

Degenerating and regenerating fibers were counted in 10 high powered fields (HPFs) of H&E stained sections per muscle from at least 6 mice per time point and identified as previously described (Pedraza et al., 2012, Proc Natl Acad Sci USA 109:4245-4250). Briefly, degenerating nuclei were counted based on discoloration and more than 3 centrally located nuclei whereas regenerating fibers had normal coloration and 2 or less centrally located nuclei. Transcription factor analysis was performed by immunohistochemistry and identification of positively stained nuclei. Nuclei were counted in 10 HPFs per muscle from at least 6 mice per time point. Vessel diameter was quantitated from at least 10 HPFs of H&E stained sections. Diameter was measured from the narrowest point of each vessel using ImageJ software. Fluorescent analyses was carried out on serial tissue sections (8 μm) using WGA Alexa Fluor 594 to label myofiber membranes, the auto-fluorescence of collagen (green) to visualize individual myofibers and the auto-fluorescence of red blood cells (yellow) to identify blood vessels. DAPI was used to stain nuclei.

### In Vivo Muscle Strength Analysis

Contractile function (i.e., torque-frequency relationship) of the left anterior crural muscles was measured in vivo using similar methodology as previously described for mice (Kalka et al., 2000, Proc Natl Acad Sci USA 97:3422-3427; Boutilier, 2001, J Exp Biol 204:3171-3181) After rats were anesthetized (2-2.5% isoflurane), the left hindlimb was aseptically prepared. The rat was then placed on a heated platform. The left knee was clamped and the left foot was secured to a custom-made foot plate that is attached to the shaft of an Aurora Scientific 305C-LR-FP servomotor, which in turn was controlled using a PC. Sterilized percutaneous needle electrodes were inserted through the skin for stimulation of the left common peroneal nerve. Electrical stimulus was applied using a Grass S88 stimulator with a constant current SIU (Grass Model PSIU6). Stimulation voltage and needle electrode placement were optimized first with a series of twitch contractions at 1 hz and then with 5 to 10 isometric contractions (400 ms train of 0.05-0.1 ms pulses at 100 Hz). Contractile function of the anterior crural muscles was assessed by measuring maximal isometric torque as a function of stimulation frequency (1-200 Hz). For real-time analysis of torque and length changes, voltage outputs were sampled at 4000 Hz, converted to a digital signal using an A/D board (National Instruments PCI 6221) and recorded using a PC loaded with a custom-made Labview®-based program (provided by the U.S. Army Institute of Surgical Research).

### Statistics

Each functional and morphological measure was compared among groups using a one-way ANOVA. A value of P<0.05 was considered to indicate statistical significance. In the event of a significant ANOVA, Bonferroni posttests for in vitro analyses and post-hoc means comparison testing with Fisher's LSD correction for in vivo analyses were performed. Statistical analyses for in vitro analyses were performed using Graphpad Prism 5. In vivo analyses were performed using SPSS 12.0

The results of the experiments are now described.

### Effect of ADA Inhibitors on Cell Survival

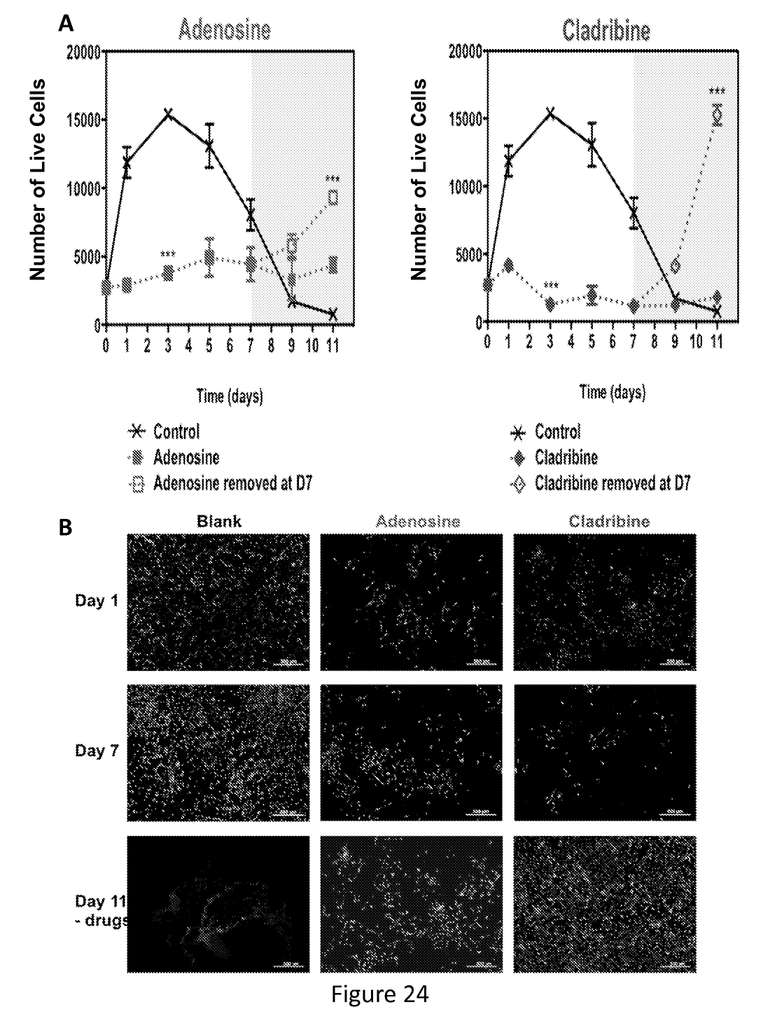

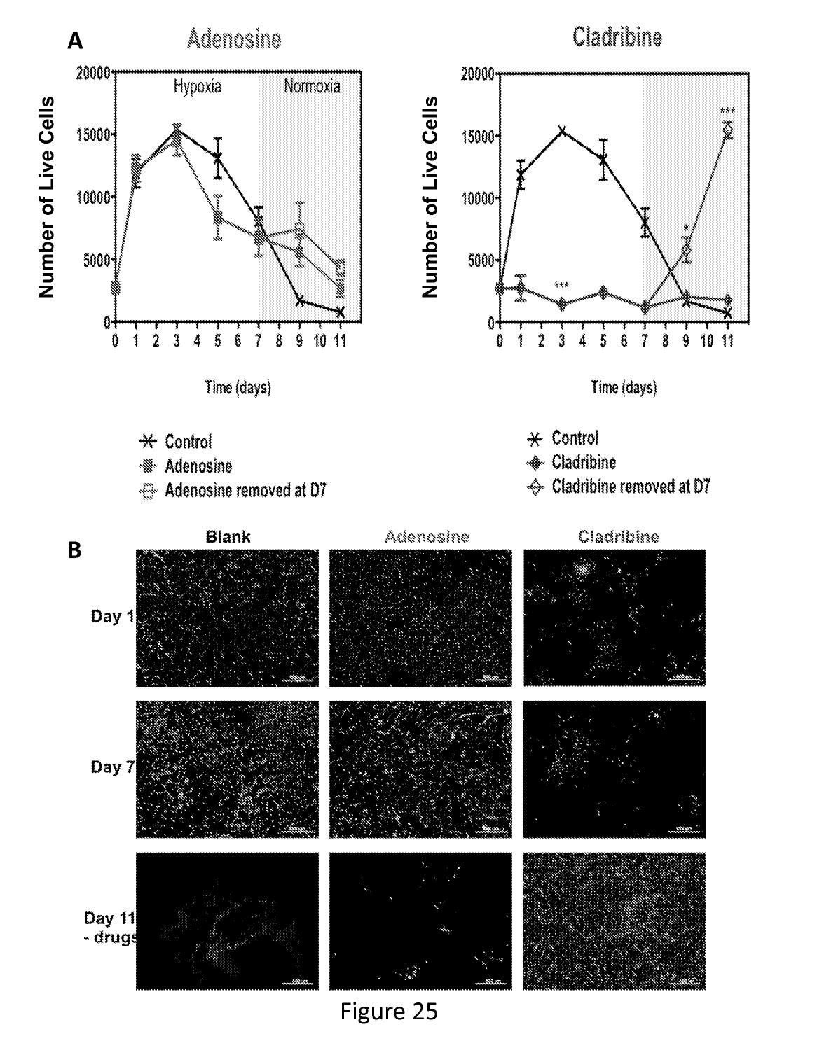

The effect of ADA inhibitors on cell survival under hypoxic condition was evaluated and compared with the effect of adenosine on cell survival under hypoxic condition. Adenosine was able to maintain cell population significantly lower than the no treatment control when used alone. When the cells were moved to normoxic condition and the drug was removed, cells started to regain their normal proliferation (FIG. 24). When treated with adenosine deaminase (ADA), adenosine-treated cells showed similar proliferation profile compared to no treatment control in hypoxic condition and failed to revive after the hypoxia and drug was removed (FIG. 25). On the other hand, cladribine-treated cells maintained minimal population with significant difference when compared to control for 7 days in hypoxic condition regardless of the presence of ADA. When the drug and hypoxia were removed, the cells regained their proliferation capability.

### Effect on Cell Metabolism

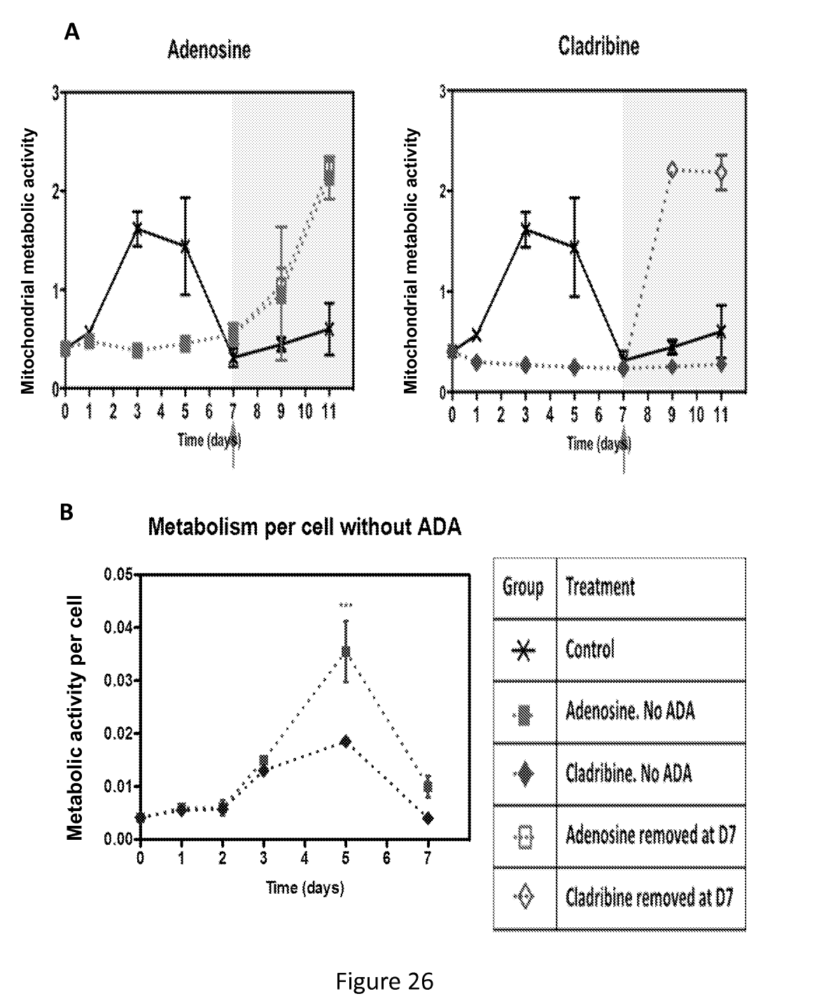

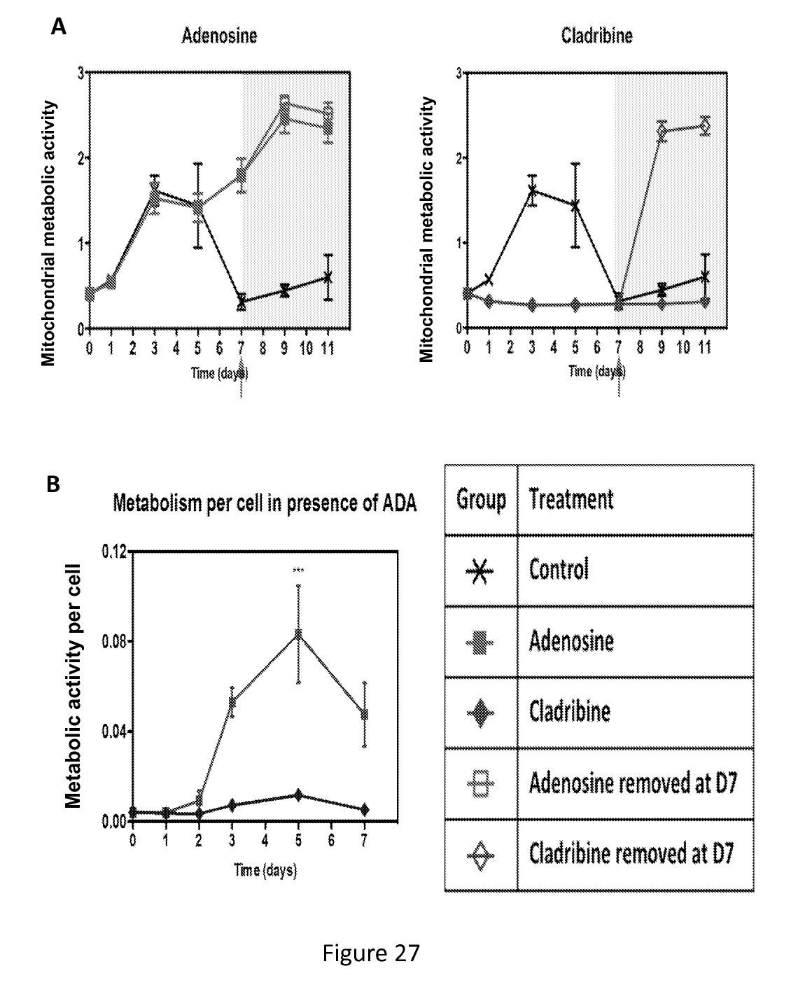

The metabolic downregulation effect of ADA inhibitors was evaluated in vitro to investigate the cause of longer cell survival. When used alone, adenosine minimized cellular metabolism. Once the drug and hypoxia were removed, the cells recovered normal metabolism (FIG. 26). However, adenosine treated with ADA, failed to reduce cellular metabolism and showed increased metabolic activity when compared to control (FIG. 27). Meanwhile, the cells treated with cladribine were kept in hypometabolic steady state without being affected by ADA. And when the hypoxia and drug were removed, the cladribine-cells regained metabolism activity. Under the effect of hypoxia, cells treated with cladribine showed significantly lower metabolic activity per cell in the presence and absence of ADA than adenosine treated cells. Although not wishing to be bound by any particular theory, these results suggest that the cells treated with cladribine possess metabolic downregulating potential.

### In Vitro Ischemic TA Muscle

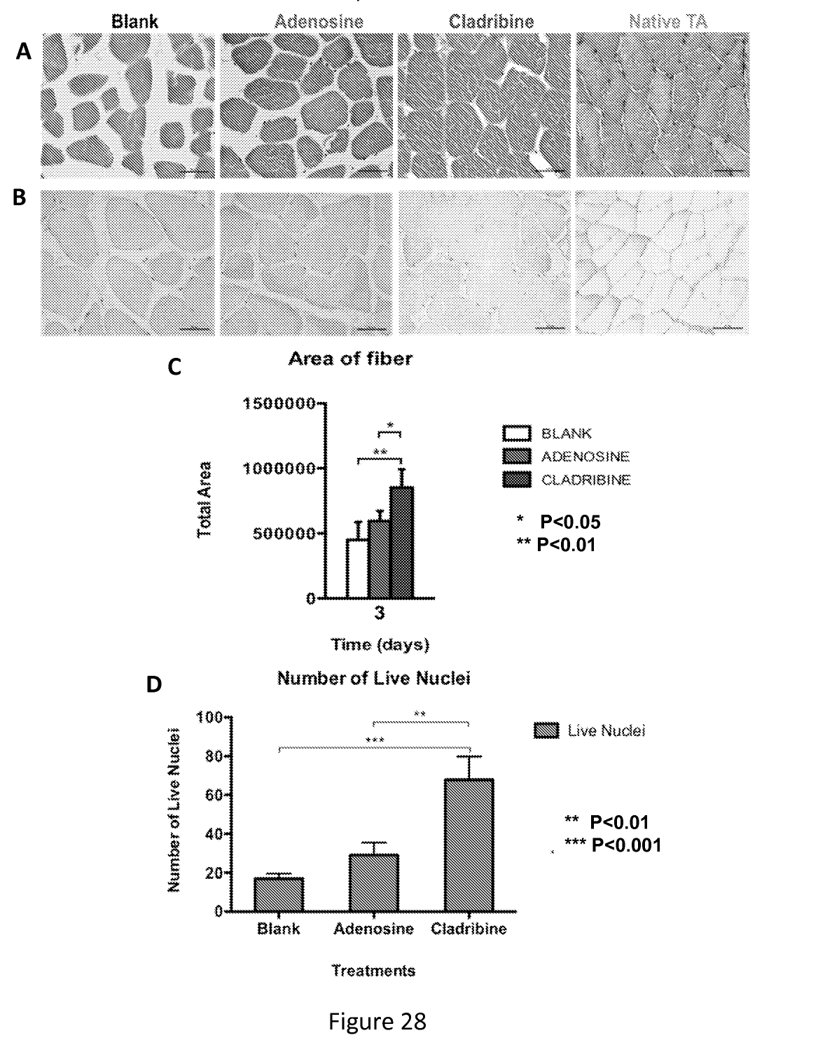

To evaluate tissue survival efficiency, rat tibialis anterior (TA) muscles were harvested, injected with adenosine or cladribine, and incubated for 3 days. The muscle is considered to be under ischemic condition due to the lack of blood supply. In the histological analyses, no treatment group showed disruption in the structure of muscle fibers. While adenosine-injected muscles showed slightly increased live nuclei, cladribine-injected muscles showed the most preserved muscle structure, significantly increased area of fiber and increased number of live nuclei when compared to control as well as adenosine-treated muscles (FIG. 28).

### Effect of Cladribine on Protecting Ischemic Tissue Using Two Rodent Ischemic Models

The effect of cladribine on tissue survival was evaluated using the following two ischemic rodent models.

Skin flap model: Histological analyses revealed that the distal necrosis in flaps treated with cladribine was clearly reduced compared with that in the control group in terms of skin discoloration at day 3 post-operation (FIG. 29B). In the histological examination (FIG. 29C), there was a clear survival benefit for the cladribine-treated group with better preservation of general tissue architecture, thickness of the skin and epidermis height. The control group had already lost much of the height in the stratified layer and dermis. In the control group, disruption of tissue architecture and indistinguishable transitions between the layers and an eosin positive mass replacing the dermis was observed. In contrast, the cladribine-treated group showed a slower progression with remaining defined layers and intact epidermis.

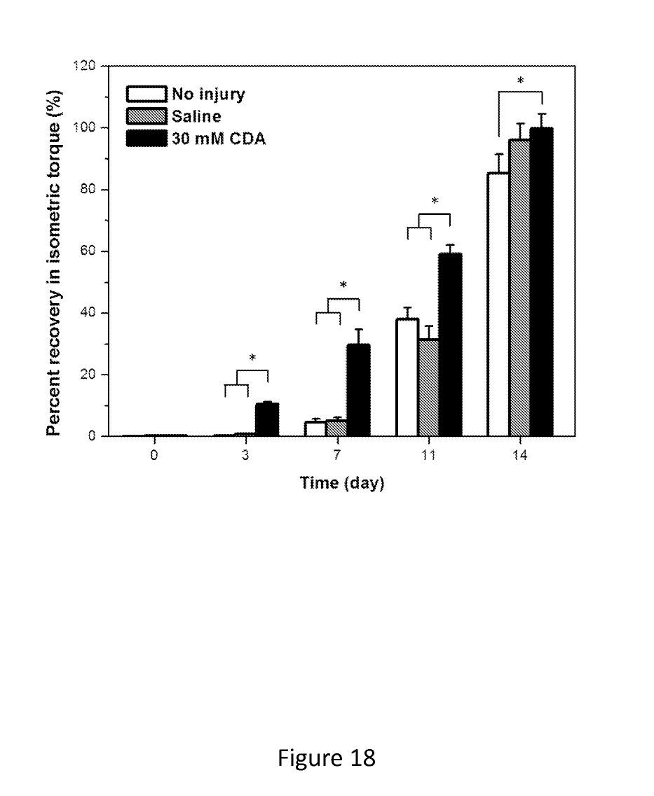

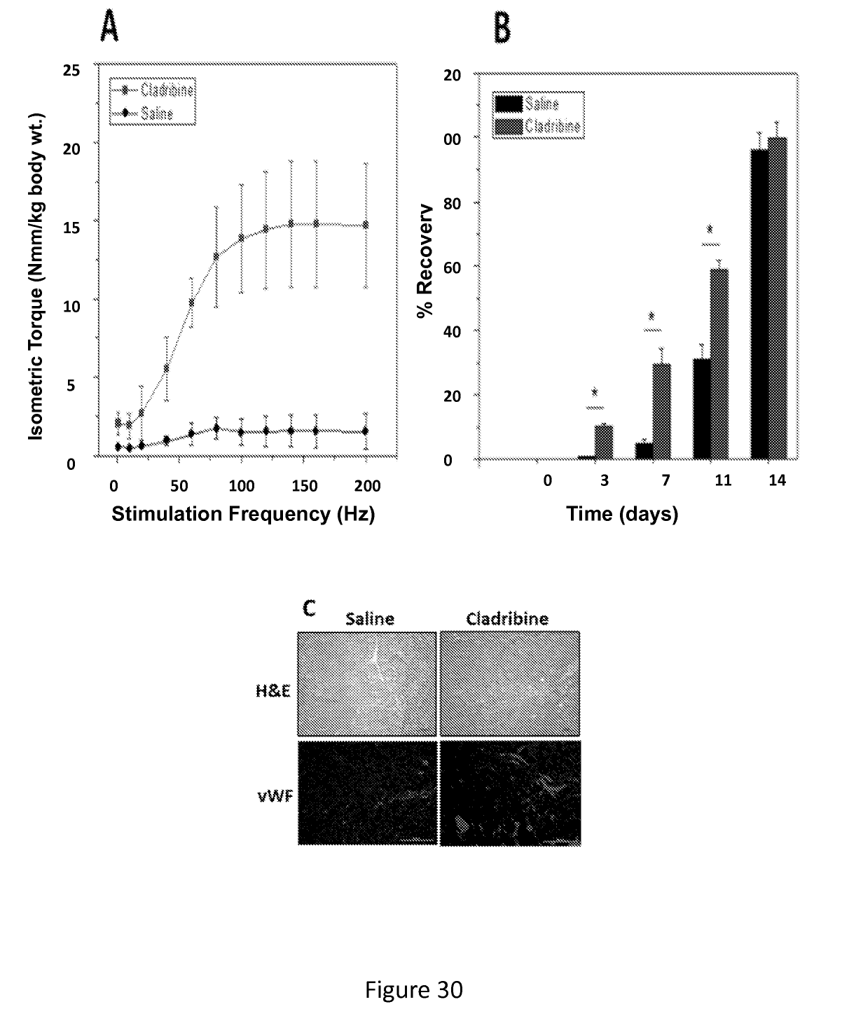

Compartment syndrome model: Compression injury that simulated compartment syndrome injury resulted in a decrease in the functional capacity of the affected TA muscles 7 days after injury (FIG. 30A). However, the TA muscle twitch isometric torque, as determined via neural stimulation, significantly increased in the cladribine-treated group. FIG. 30B shows the percent recovery based on the tetanic outcome at 100 Hz of the intact animals. When cladribine treated, a significant acceleration of recovery in muscle function was observed compared with that of the control group (FIGS. 18, 30B). Qualitative analyses on H&E and immuostainings of both groups were also performed (FIG. 30C). Edema and swelling in the saline treated group was evident, and the cladribine treated group showed more newly forming vessels, indicating more neovascularization.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of increasing the viability of a cell under a hypoxic condition, comprising contacting the cell with an effective amount of adenosine or a derivative thereof to reduce the oxygen demand of the cell.

2. The method of claim 1, wherein the effective amount of adenosine or a derivative thereof downregulates the metabolic rate of the cell.

3. The method of claim 1, wherein contacting the cell with an effective amount of adenosine or a derivative thereof further results in a steady state of cellular metabolic activity.

4. The method of claim 1, wherein the cell resumes a normal proliferation rate when the adenosine or a derivative thereof is removed from the cell.

5. The method of claim 1, wherein the cell is a myoblast.

6. The method of claim 5, wherein the cell is a murine myoblast.

7. The method of claim 5, wherein the cell is a human myoblast.

8. A method of increasing cellular survival in a tissue-engineered construct during vasculogenesis, comprising administering an effective amount of adenosine or a derivative thereof to the cells in the tissue-engineered construct to downregulate the metabolic rate of the cells until host vascularization is established.

9. A method of prolonging the survival of an implanted cell that is under a hypoxic condition in a host, comprising contacting the cell with an effective amount of adenosine or a derivative thereof to reduce the oxygen demand of the cell until host neovascularization is achieved.

10. The method of claim 9, wherein the effective amount of adenosine or a derivative thereof downregulates the metabolic rate of the cell.

11. The method of claim 9, wherein contacting the cell with an effective amount of adenosine or a derivative thereof further results in a steady state of cellular metabolic activity.

12. The method of claim 9, wherein the hypoxic cell resumes a normal proliferation rate when the effects of the adenosine or a derivative thereof are removed.

13. The method of claim 9, wherein the cell is a myoblast.

14. The method of claim 13, wherein the cell is a murine myoblast.

15. The method of claim 13, wherein the cell is a human myoblast.

16. A method of increasing the viability of a cell under a hypoxic condition, comprising prolonging the availability of adenosine or a derivative thereof in the cell by contacting the cell with an effective amount of a purine metabolic enzyme inhibitor, such that the activity of the inhibited purine metabolic enzyme is reduced, and wherein the prolonged availability of adenosine or a derivative thereof results in a reduction of the oxygen demand of the cell.

17. The method of claim 16, wherein the purine metabolic enzyme is adenosine deaminse.

18. The method of claim 17, wherein the purine metabolic enzyme inhibitor is selected from the group consisting of fludarabine phosphate, pentostatin, cladribine, coformycin, 2′-deoxycoformycin, erythro-9-(2-hydroxy-3-nonyl)adenine (EHNA), 9′-hydroxy-EHNA, 9′-chloro-EHNA, 9′-phthalimido-EHNA, 8′,9′-didehydro-EHNA, 1-deaza-EHNA, 3-deaza-EHNA, adechlorin, adecypenol, 1-deazaadenosine, 1-deaza-2′-deoxyadenosine, 3′-deoxy-1-deazaadenosine, 2′,3′-dideoxy-1-deazaadenosine, (2S,3R)-3-(6-amino-9H-purin-9-yl)-7-(o-tolyl)heptan-2-ol, erythro-9-(2-hydroxy-3-nonyl)-1,2,4-triazole, erythro-9-(2-hydroxy-3-nonyl)-1,2,4-triazole-3-carboxamide, kampherol, quercitin, 2-[4-[4,4-bis(4-fluorophenyl)butyl]piperazin-1-yl]-N-(2,6-dimethylphenyl)acetamide, dipyridamole, trazodone, or phenylbutazone.

19. The method of claim 18, wherein the purine metabolic enzyme inhibitor is cladribine.

20. The method of claim 16, wherein the cell resumes a normal proliferation rate when the purine metabolism enzyme inhibitor is removed from the cell.

21. A method of increasing cellular survival in a tissue-engineered construct during vasculogenesis, comprising administering an effective amount of purine metabolism enzyme inhibitor to the cells in the tissue-engineered construct to prolong the availability of adenosine or a derivative thereof present in the cells, wherein the prolonged availability of adenosine or a derivative thereof down-regulates the metabolic rate of the cells until host vascularization is established.

22. A method of prolonging the survival of an implanted cell that is under a hypoxic condition in a host, comprising contacting the cell with an effective amount of purine metabolism enzyme inhibitor to prolong the availability of adenosine or a derivative thereof, wherein the prolonged availability of adenosine or a derivative thereof reduces the oxygen demand of the implanted cell.

23. The method of claim 22, wherein the hypoxic cell resumes a normal proliferation rate when the effects of purine metabolism enzyme inhibitor are removed.
